(19) United States
(12) Patent Application Publication
(10) Pub. No.: US 2023/0333038 A1
(43) Pub. Date: Oct. 19, 2023

(54) MASK-BASED DIAGNOSTIC DEVICE AND WAFER-LEVEL FUNCTIONALIZATION OF A PACKAGED SEMICONDUCTOR BIOSENSOR

(71) Applicant: Diagmetrics, Inc., Madison, CT (US)

(72) Inventors: John James Daniels, Madison, CT (US); Rabah Boukherroub, Villeneuve d'Ascq (FR)

(21) Appl. No.: 17/864,343

(22) Filed: Jul. 13, 2022

Related U.S. Application Data
(60) Provisional application No. 63/331,841, filed on Apr. 17, 2022.

(51) Int. Cl. G01N 27/12; H01L 29/66; A61B 5/00

(52) U.S. Cl. CPC G01N 27/128; H01L 29/66045; A61B 5/6803; A61B 2010/0087

(57) ABSTRACT

A mask-based diagnostic device, includes a face mask, where an inside of the face mask defines during use a confined local environment that includes breath vapor exhaled from the lungs of a user. An exhaled breath condensate (EBC) collector has a condensate forming surface for converting exhaled breath vapor into an EBC liquid sample. The EBC collector includes a thermal mass cooled before use to a condensation forming temperature less than a confined environment temperature of the confined environment inside of the face mask, and an EBC testing unit for testing the EBC sample for a target molecule, where the EBC sample contains water and the target molecule, the EBC testing unit includes a printed circuit board supporting a semiconductor packaged electronic biosensor in electrical communication with power, analysis and communications electronics, a fluid conductor for conducting the EBC sample to the electronic biosensor.

### CROSS-REFERENCE TO RELATED APPLICATIONS

This US Utility patent application claims the benefit of priority of U.S. provisional patent application No. 63/331,841, filed on Apr. 17, 2022, entitled “Mask-Based Diagnostic Device and Package Semiconductor Biosensor, the teachings of which are incorporated by reference herein in its entirety.

### BACKGROUND OF THE INVENTION

The exemplary and non-limiting embodiments relate generally to diagnostic systems, methods, devices and computer programs and, more specifically, relate to digital and analog diagnostic devices for detecting a biomarker of a biological agent such as a coronavirus, lung cancer, tuberculosis, asthma, and other respiratory ailments and conditions, and/or blood borne biomarkers and other biomarkers that are present in the exhaled breath of a test subject.

The present invention also pertains to a device architecture, specific-use applications, and computer algorithms used to detect biometric parameters for the treatment and monitoring of physiological conditions in humans and animals by testing for biomarkers in exhaled breath, sweat, blood, urine, feces, gastro-intestinal lavage, interstitial fluid, mucus, saliva, or other bodily fluid.

This section is intended to provide a background or context to the exemplary embodiments of the invention as recited in the claims. The description herein may include concepts that could be pursued but are not necessarily ones that have been previously conceived, implemented or described.

Therefore, unless otherwise indicated herein, what is described in this section is not prior art to the description and claims in this application and is not admitted to being prior art by inclusion in this section.

Testing for biomarkers that indicate exposure, infection, progression and recovery from a disease condition, such as COVID-19 can be used to screen individuals for infection and help slow the spread of the virus. For example, protein and RNA testing for active virus shows who is currently contagious. Antibody testing can be used to find the members of a population that have recovered from the virus.

Diagnostics of SARS-CoV-2 infection using real-time reverse-transcription polymerase chain reaction (RT-PCR) on nasopharyngeal swabs is now well-established, with saliva-based testing being lately more widely implemented for being more adapted for self-testing approaches. The procedure to obtain nasal swab samples is not only uncomfortable, but also often requires specialized personnel with risk of contaminating the person performing the test. Saliva tests have the advantage of being simpler to perform, less invasive with limited risks and RT-PCR on saliva specimens has becoming more widely implemented. The viscose nature of saliva together with the presence of saliva proteases, responsible for the proteolytic activity of saliva, make the direct application of saliva samples challenging. It is well known that the major mechanisms of COVID-19 spread are airborne and contact infections primarily due to aerosol droplets expelled from the lungs and airways of infected persons. There is therefore a growing need for sample collection by patients themselves and a simple to use testing system that can detect a target biomarker indicative of a pathogenic infection from a biosample obtained from the lungs and airways.

The exemplary and non-limiting embodiments relate generally to diagnostic systems, methods, devices and computer programs and, more specifically, relate to digital and analog diagnostic devices for detecting a biomarker of a biological agent such as a coronavirus, lung cancer, tuberculosis, asthma, and other respiratory ailments and conditions, and/or blood borne biomarkers and other biomarkers that are present in the exhaled breath of a test subject.

The present invention also pertains to a device architecture, specific-use applications, and computer algorithms used to detect biometric parameters for the treatment and monitoring of physiological conditions in humans and animals.

### SUMMARY OF THE INVENTION

The below summary section is intended to be merely exemplary and non-limiting. The foregoing and other problems are overcome, and other advantages are realized, by the use of the exemplary embodiments of this invention.

In one aspect, a mask-based diagnostic device, includes a face mask, where an inside of the face mask defines during use a confined local environment, where the local environment includes breath vapor exhaled from the lungs of a user, an exhaled breath condensate (EBC) collector having a condensate forming surface for converting exhaled breath vapor into an EBC liquid sample, the EBC collector including a thermal mass cooled before use so that during use the condensate forming surface is at a condensation forming temperature less than a confined environment temperature of the confined environment inside of the face mask, and an EBC testing unit for testing the EBC sample for a target molecule, where the EBC sample contains water and the target molecule, the EBC testing unit includes a printed circuit board supporting a semiconductor packaged electronic biosensor in electrical communication with power, analysis and communications electronics, a fluid conductor for conducting the EBC sample to the electronic biosensor, a fluid detector onboard the printed circuit board for detecting at least one of a start of fluid conduction through the fluid conductor in a flow path before the electronic biosensor and an end of fluid conduction in the flow path after the electronic biosensor, the analysis electronics for detecting the target molecule dependent on an electrical signal received from the electronic biosensor, and the communications electronics for communicating the detection of the target molecule.

In another aspect, a mask-based diagnostic device with removable and recyclable components, includes a support member configured and dimensioned to removably fit into a face mask, where an inside of the face mask defines a confined local environment, where during use the local environment includes breath vapor exhaled from the lungs of a user, an EBC collector supported on the support member, disposed during use within the defined volume, and having a condensate forming surface for converting the exhaled breath vapor into an exhaled breath condensate (EBC) sample, the EBC collector including a thermal mass cooled before use so that during use the condensate forming surface is at a condensation forming temperature less than a confined environment temperature of the confined local environment inside of the face mask, and an attachment member for removably attaching the support member and EBC collector to the inside of the face mask.

In another aspect, a liquid sample concentrator for a mask-based diagnostic concentrator, includes a filter member having an obverse side and a reverse side, where the filter member has a minimum particle filtering size, a super absorbent polymer (SAP) particulate disposed on the obverse side, where during use a fluid sample includes a carrier fluid sample containing a target molecule contacts the reverse side and flows along a length of the filter member where a portion of the fluid sample flows through the filter member to wet the SAP, where the super absorbent polymer selectively absorbs the carrier fluid sample to concentrate the target molecule in the carrier fluid sample flowing along the length.

In another aspect, a method for making a semiconductor biosensor, includes the steps of i) Providing a semiconductor substrate wafer of one conductivity type, ii) Forming a plurality of semiconductor device regions in the semiconductor substrate wafer, each semiconductor device region includes at least a source region and a drain region defining there between a channel region of the one conductivity type with the source region and the drain region of an opposite conductivity type, iii) Forming a detection area over the channel region, the detection area including a charge transfer layer, iv) Immobilizing capture molecules on the charge transfer layer of at least a portion of the plurality of semiconductor device regions, and v) Separating individual semiconductor devices from the semiconductor substrate wafer, each includes at least one semiconductor device region of the plurality of semiconductor device regions, where the step of separating is performed after the step immobilizing.

In another aspect, a method, includes the steps of i) Providing a semiconductor wafer, ii) Forming device regions includes a source, drain and a channel region, iii) Forming at least one of an insulator layer and a dielectric layer over at least the channel region, iv) Forming a detection area including a charge transfer layer over said at least one of an insulator layer and a dielectric layer, v) Immobilizing capture molecules on the charge transfer layer, and vi) After immobilizing the capture molecules separating individual semiconductor devices from the semiconductor wafer.

In another aspect, a method, includes the steps of i) Providing a semiconductor wafer, ii) Forming device regions each includes a source, a drain and at least one channel region, iii) Forming at least one of an insulator layer and a dielectric layer over each channel region, iv) Forming a detection area including a charge transfer layer over said at least one of an insulator layer and a dielectric layer, v) Immobilizing capture molecules on the charge transfer layer, where immobilizing capture molecules includes the steps of a) immobilizing activatable linker molecules on the charge transfer layers, and b) disposing a capture molecule carrier fluid containing the capture molecules as free-floating capture molecules over a top surface of the semiconductor substrate wafer covering the plurality of device regions, where prior to activation the activatable linker molecules are relatively less receptive to binding to the free-floating capture molecules. The method also includes c) selectively activating the activatable linker molecules to form activated linker molecules immobilized at some of the charge transfer layers, the activated linker molecules bind with the free-floating capture molecules. The method also includes d) binding the capture molecules to the activated linker molecules.

In another aspect, a method, includes the steps of i) Providing a semiconductor wafer, ii) Forming device regions each includes a source, a drain and at least one channel region, iii) Forming a gate oxide layer over each channel region, iv) Forming a detection area including a charge transfer layer over the gate oxide layer, v) Immobilizing capture molecules on the charge transfer layer. The step of immobilizing capture molecules includes the steps of a) immobilizing at least a first set of activatable linker molecules and a second set of activatable linker molecules on the charge transfer layer of device region. Each respective first set and second set of activatable linker molecules being activated for binding by a different corresponding first wavelength of linker-activating radiation and second wavelength of linker-activating radiation. The method also includes b) disposing over a surface of the semiconductor substrate wafer covering the plurality of device regions a capture molecule carrier fluid containing at least a first set of activatable capture molecules and a second set of activatable capture molecules as free-floating activatable capture molecules. Each respective first set and second set of activatable capture molecules being activated for binding by a different corresponding first wavelength of capture molecule-activating radiation and second wavelength of capture molecule-activating radiation. The method also includes c) selectively irradiating the surface of the semiconductor wafer with a first pattern of radiation including the first wavelength of linker-activating radiation and the first wavelength of capture molecule-activating radiation to bind a first set of activated capture molecules to a first set of activated linker molecules. The method also includes d) selectively irradiating the surface of the semiconductor wafer with a second pattern of radiation including the second wavelength of linker-activating radiation and the second wavelength of capture molecule-activating radiation to bind a second set of activated capture molecules to a second set of activated linker molecules.

### BRIEF DESCRIPTION OF THE DRAWINGS

To easily identify the discussion of any particular element or act, the most significant digit or digits in a reference number refer to the figure number in which that element is first introduced.

FIG. 1 illustrates an embodiment of the mask-based diagnostic device having an EBC collector and sample collection pool that is retrofit into a pre-existing face mask;

FIG. 2 illustrates an EBC collector with a centrally mounted EBC testing unit.

FIG. 3 illustrates an assembled EBC testing unit.

FIG. 4 is an exploded view showing components of an EBC testing unit.

FIG. 5 is an exploded view showing the components of the EBC collector.

FIG. 6 is a block diagram of the electronic circuit of an EBC test unit.

FIG. 7 shows components of a liquid sample concentrator with a close up view showing a SAP particulate embedded on adhesive.

FIG. 8 shows a liquid sample collector provided in a collection pool of an EBC collector.

FIG. 9 shows a removable and recyclable EBC collector.

FIG. 10 shows the removable and recyclable EBC collector retrofit into a pre-existing face mask.

FIG. 11 shows a removable and recyclable EBC testing unit centrally mounted on an EBC collector and in fluid communication with the collection pool.

FIG. 12 shows a sealing member sealing the collection pool of a removable and recyclable EBC collector fixed to a disposable face mask.

FIG. 13 shows the sealed EBC collector removed from the disposable face mask.

FIG. 14 shows the components of a liquid sample concentrator.

FIG. 15 shows the removed EBC collector with the sample concentrator disposed in the collection pool being sealed.

FIG. 16 illustrates a liquid sample concentrator having a low surface energy substrate, fluid conductor and selective water absorber.

FIG. 17 is a close up showing grooves of the low surface energy substrate for channeling a concentrated liquid sample to the collection pool in fluid communication with an EBC testing unit.

FIG. 18 illustrates an EBC collector with a centrally mounted EBC testing unit and a pair of liquid sample concentrators.

FIG. 19 shows under black light a water carrier fluid with florescent nanoparticles being disposed onto a sample concentrator having filter paper covering a layer of un-swelled SAP particulate embedded on an adhesive sheet.

FIG. 20 shows swelled SAP particulate after absorbing water with florescent nanoparticles.

FIG. 21 shows an un-rinsed portion of the swelled SAP particulate with florescent nanoparticles.

FIG. 22 shows the portion of swelled SAP particulate after rinsing away the florescent nanoparticles.

FIG. 23 illustrates the EBC testing unit showing a dissolvable surfactant film disposed in the fluid flow path before the sample flow reaches the electronic biosensor.

FIG. 24 illustrates a version of the EBC testing unit showing two parallel biosensors for simultaneously receiving the fluid sample at the detection area of each biosensor.

FIG. 25 is an isolated view of a semiconductor double in-line packaged (DIP)biosensor for incorporation in a printed circuit board.

FIG. 26 illustrates the lid of a table top EBC testing unit.

FIG. 27 illustrates the housing in which sits an EBC testing unit printed circuit board (PCB).

FIG. 28 illustrates an assembled table top EBC testing unit showing a testing well for reaching a liquid sample.

FIG. 29 illustrates a PCB board with a coin cell battery and DIP socket for receiving the semiconductor DIP packaged biosensor.

FIG. 30 illustrates the PCB board sitting in the housing with an o-ring for sealing the detection well of the electronic biosensor to the lid of the table top EBC testing unit.

FIG. 31 illustrates the table top version of the EBC test unit showing a dropper for disposing a liquid sample into a test well in the lid.

FIG. 32 illustrates the steps of making a semiconductor wafer of graphene field effect transistor biosensors with a protection layer patterned in place over the detection interfaces of the g-FET biosensors.

FIG. 33 is a flow chart for making a semiconductor wafer of graphene field effect transistor biosensors with capture molecules immobilized on the detection interface prior to dicing the semiconductor wafer.

FIG. 34 is a flow chart for making the semiconductor wafer of graphene field effect transistor biosensors with capture molecules immobilized onto selectively activatable linker molecules.

FIG. 35 illustrates the steps for making the semiconductor wafer of graphene field effect transistor biosensors with selectively immobilizing a second type of capture molecules without disturbing the capture molecules previously immobilized on the first type of functionalized biosensors, with a dissolvable protection layer formed over the surface of the wafer.

FIG. 36 illustrates the steps for making the semiconductor wafer of graphene field effect transistor biosensors with selectively immobilizing a second type of capture molecules without disturbing the capture molecules previously immobilized on the first type of functionalized biosensors, with a dissolvable protection layer formed over the surface of the wafer.

FIG. 37 illustrates a diced semiconductor wafer of g-FET biosensors with an individual g-FET biosensor being picked by a nozzle of a conventional pick and place machine.

FIG. 38 illustrates a work holder for holding a plurality of semiconductor packaged g-FET biosensors for batch functionalization using an automatic dispensing system.

FIG. 39 illustrates a wire bonding process for packaging a g-FET biosensor bare die in a conventional DIP semiconductor package.

FIG. 40 illustrates a semiconductor DIP packaged g-FET biosensor with a detection well for providing a liquid sample to the detection interface of the g-FET.

FIG. 41 is an exploded view illustrating a non-wire bonded alternative construction for connecting a bare die g-FET biosensor to a microprocessor PCB.

FIG. 42 shows a prototype non-wire bonded alternative construction of a bare die g-FET biosensor connected to a microprocessor PCB.

FIG. 43 shows a smartphone having an application (APP) for communicating with the EBC testing unit and conveying information to the phone user and/or to a remote network server.

FIG. 44 shows screens of a smartphone APP providing a graphical user interface for conveying the steps for conducting a test using a mask-based diagnostic device and receiving information indicating the test results.

FIG. 45 illustrates activatable linker molecules before activation, with free-floating capture molecules in a liquid carrier.

FIG. 46 illustrates some activatable linker being selectively activated by photo radiation for binding to the capture molecules.

FIG. 47 illustrates the activated linker binding with free-floating capture molecules.

FIG. 48 illustrates different capture molecules for detecting different capture molecules and bond to activatable linkers.

FIG. 49 illustrates the steps of selectively binding a first sub-set of capture molecules to activatable linker molecules.

FIG. 50 illustrates the steps of selectively binding a second sub-set of capture molecules to activatable linker molecules.

FIG. 51 illustrates a flow chart for making a semiconductor wafer of g-FETs with different capture molecules bond to activatable linker molecules at the detection area of different g-FETs.

FIG. 52 illustrates activatable capture molecules, activated capture molecules, activatable linker molecules and activated linker molecules used for wafer-level functionalization of semiconductor biosensors.

FIG. 53 illustrates the steps for selectively binding a first set of activated capture molecules to a first set of activated linker molecules.

FIG. 54 illustrates the steps for selectively binding a second set of activated capture molecules to a second set of activated linker molecules, to form a bare die semiconductor device with two or more separately functionalized g-FET biosensors.

FIG. 55 is a flow chart showing the steps for selectively immobilizing different respective capture molecules on different corresponding g-FETs.

FIG. 56 illustrates a single bare die wire bonded for semiconductor packaging and having multiple and differently functionalized g-FET biosensors.

FIG. 57 illustrates bare die biosensor having g-FET devices functionalized for FluA/FluB/SARS virus testing.

FIG. 58 illustrates a bare die biosensor having multiple and differently functionalized g-FET biosensors for detecting lung cancer.

FIG. 59 illustrates a bare die biosensor having twelve functionalized g-FET biosensors for detecting one or more diseases, health conditions and/or environmental conditions.

FIG. 60 illustrates the cross-linking of pyrenebutyric acid N-hydroxysuccinimide ester (PBASE) with an aminated nanoCLAMP capture molecule to form a pyrene-tagged nanoCLAMP (PTNC) capture molecule conjugate, and then a one-step process for immobilizing the PTNC capture molecule conjugate on a graphene charge transfer layer.

FIG. 61 illustrates bare die g-FET biosensors diced from the wafer after the one-step process for immobilizing the PTNC capture molecule conjugate and the formation of a protection layer at the wafer level.

FIG. 62 illustrates the steps of making a semiconductor wafer of graphene field effect transistor biosensors with a one-step process for immobilizing PTNC capture molecule conjugates and the formation of a protection layer at the wafer level.

FIG. 63 is a flowchart of the steps for making a semiconductor wafer of g-FET biosensors where capture molecule conjugates are immobilized on graphene charge transfer layers at the wafer level.

### DETAILED DESCRIPTION OF THE ILLUSTRATED EMBODIMENT

Below are provided further descriptions of various non-limiting, exemplary embodiments. The exemplary embodiments of the invention, such as those described immediately below, may be implemented, practiced or utilized in any combination (e.g., any combination that is suitable, practicable and/or feasible) and are not limited only to those combinations described herein and/or included in the appended claims.

The word “exemplary” is used herein to mean “serving as an example, instance, or illustration.” Any embodiment described herein as “exemplary” is not necessarily to be construed as preferred or advantageous over other embodiments. In any case, all of the embodiments described in this Detailed Description are exemplary embodiments provided to enable persons skilled in the art to make or use the invention and not to limit the scope of the invention which is defined by the claims.

Many configurations, embodiments, methods of manufacture, algorithms, electronic circuits, microprocessors, memory and computer software product combinations, networking strategies, database structures and uses, and other aspects are disclosed herein for a diagnostic or testing platform, devices, methods and systems that have a number of medical and non-medical uses.

Although embodiments are described herein for detection of biomarkers of SARS-CoV-2 virus, the systems, methods and apparatus described are not limited to any particular virus or disease, or just limited to biological use-cases. In most instances, where the term virus or COVID-19 is used, any other health or fitness related biomarker could be used instead. The description here and the drawings and claims are therefore not intended to be limited in any way to virus detection, the inventions described and claimed can be used for many diseases including lung cancer, diabetes, asthma, tuberculosis, environmental exposures, glucose, lactate, blood borne diseases and other ailments or indications of the health of the test subject. Further, the electronic biosensor, test systems, uses and methods of manufacturing described herein are not limited to the use of exhaled breath condensate. Wastewater, potable water, environmental quality samples, ambient samples and any bodily fluid can be used as the test sample. The use of aptamers and engineered capture molecules, in particular, make the inventive sensor widely useful because of the nature of engineered capture molecules such as aptamers, nanoCLAMPs, nano-bodies, engineered anti-bodies, etc., being adaptable by specific engineering design and selection to have a binding affinity that is tailored to a corresponding target analyte. Therefore, the descriptions of innovations are not intended to be limited to a particular use-case, sample medium, capture molecule, biomarker or analyte.

In immunochromatography, a capture molecule, which may be, for example, an aptamer, nanoCLAMP, naturally occurring antibody, or engineered antibody, is disposed onto a surface of a porous membrane, and a sample passes along the membrane. As described herein, the term antibody, aptamer, engineered antibody, or capture molecule is used interchangeably. In some instances, a specific type of capture molecule may be described. In the case of a lateral flow assay (LFA) type testing system, biomarkers in the sample is bound by the capture molecule which is coupled to a detector reagent. As the sample passes through the area where the capture molecule is disposed, a biomarker detector reagent complex is trapped, and a color develops that is proportional to the concentration or amount biomarker present in the sample. In the case of an electronic or electro-chemical testing system, the captured biomarker causes a detectable change in a signal obtained, typically through an electrical connection with two or more electrodes.

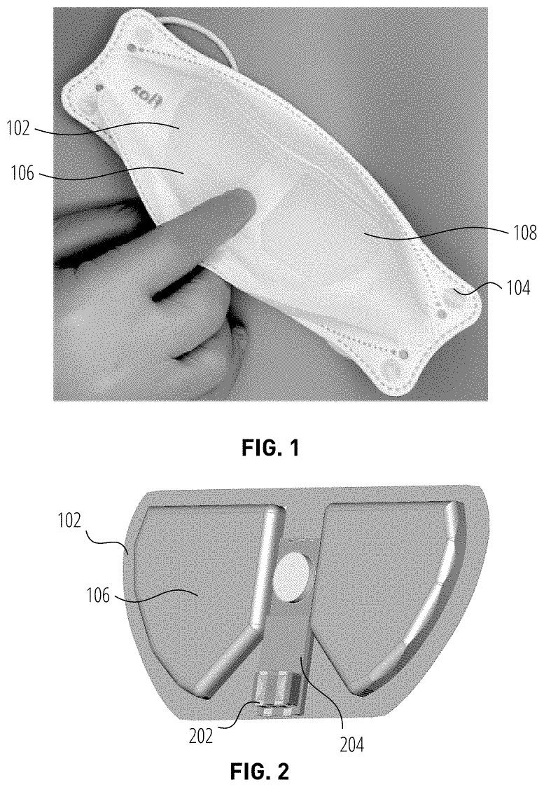

FIG. 1 shows a mask-based diagnostic device. The system comprises an EBC collector 102, a face mask 104, and a thermal mass 108. The face mask 104 defines, during use, a confined local environment. The local environment includes breath vapor exhaled from the lungs of a user. The EBC collector 102 has a condensate forming surface 106 that converts exhaled breath vapor into an EBC liquid sample. The EBC collector 102 includes a thermal mass 108 cooled before use so that during use the condensate forming surface is at use to a condensation forming temperature less than a confined environment temperature of the confined environment inside of the face mask.

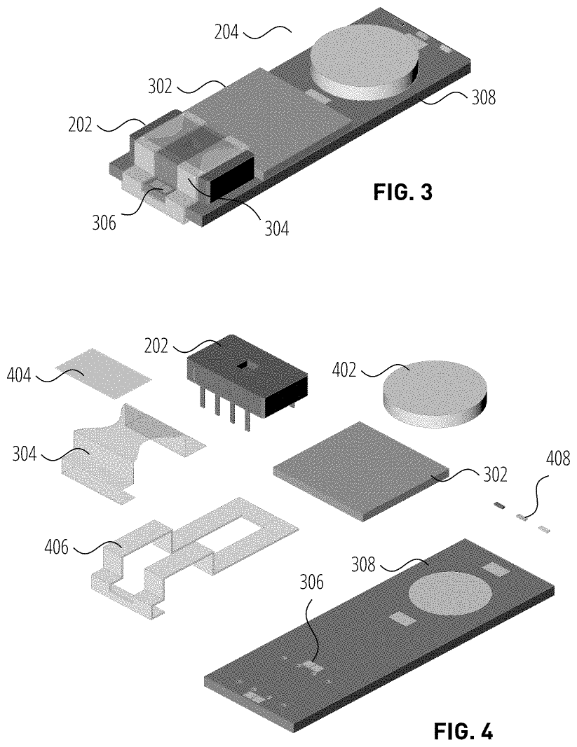

FIG. 2 illustrates an EBC collector with a centrally mounted EBC testing unit. The system comprises an EBC collector 102, a condensate forming surface 106, a biosensor 202, and an EBC testing unit 204. The EBC testing unit test the EBC sample for a target molecule, where the EBC sample contains water and the target molecule. The EBC testing unit includes a printed circuit board supporting a semiconductor packaged electronic biosensor in electrical communication with power, analysis electronics and communications electronics. A fluid conductor conducts the EBC sample to the electronic biosensor. A fluid detector onboard the printed circuit board detects at least one of a start of fluid conduction through the fluid conductor in a flow path before the electronic biosensor and an end of fluid conduction in the flow path after the electronic biosensor. The analysis electronics detects the target molecule dependent on an electrical signal received from the electronic biosensor. The communications electronics communicates the detection of the target molecule. The analysis electronics and the communications electronics, biosensor, printed circuit board, electronics, etc., can also be deployed in an different EBC testing unit format. For example, a lateral flow assay (LFA) testing unit can be utilized, with some of the EBC testing unit components changed or obviated, and other included. In the FIG. 3 illustrates an assembled EBC testing unit. The EBC testing unit includes the biosensor 202, the EBC testing unit 204, a wick 302, a fluid conductor 304, a fluid detector 306, and a printed circuit board 308. The fluid conductor conducts the EBC sample to the electronic biosensor.

FIG. 4 is an exploded view showing components of an EBC testing unit 204. The EBC testing unit includes the biosensor 202, the wick 302, the fluid conductor 304, the fluid detector 306, the printed circuit board 308, a battery 402, a light emitting diode LED 408, a microfluidic cover 404, and a microfluidic adhesive 406. The fluid detector onboard the printed circuit board detects when EBC is flowing through the fluid conductor and into or over the detection well of the biosensor. As an example, the fluid detector comprises a pair of parallel conductors on the printed circuit board with a gap of 0.02″ between the conductors that enables the detection of the presence of EBC in the section of the fluid conductor in contact with the parallel conductors. When EBC is present, an electrical signal can flow from one of the parallel conductors through the EBC to the other parallel conductor to complete an electrical circuit. In the embodiment shown in FIG. 4, two sets of fluid detectors are printed on the printed circuit board. Either or both the start of fluid conduction through the fluid conductor in a flow path before the electronic biosensor and the end of fluid conduction in the flow path after the electronic biosensor can be detected by the respective fluid detectors. The EBC testing unit also includes the printed circuit board supporting the semiconductor packaged electronic biosensor in electrical communication with power, analysis and communications electronics.

A transistor may be provided on the printed circuit board or to amplify the signal generated when EBC is present in the fluid conductor section in contact with the parallel conductors. Alternatively, a TTL Logic circuit (e.g., High(3.3v) and Low(0v) can be the pads, and switch on LEDs to detect when water hits the first and second set of liquid detection pads. The fluid detectors can be used to determine the time it takes for the EBC biosample to flow from the first to second set of parallel conductors to indicate how well the EBC collector is performing at converting breath vapor to liquid. The fluid detectors can as be used to indicate EBC is present at the detection well of the biosensor and to automatically initiate the testing process and analysis of the electrical signals generated by the biosensor. In addition, or alternatively, a physical switch can be provided on the printed circuit board to initiate the testing, and/or a smartphone or computer user-interface wirelessly connected with the microcontroller or microprocessor onboard the PCB can be used to control the test. Also, the detection of the EBC at the second fluid detector (in the EBC flow path after the biosensor) can be used to indicate if the test is invalid, for example, if the EBC flow does not reach the second fluid detector.

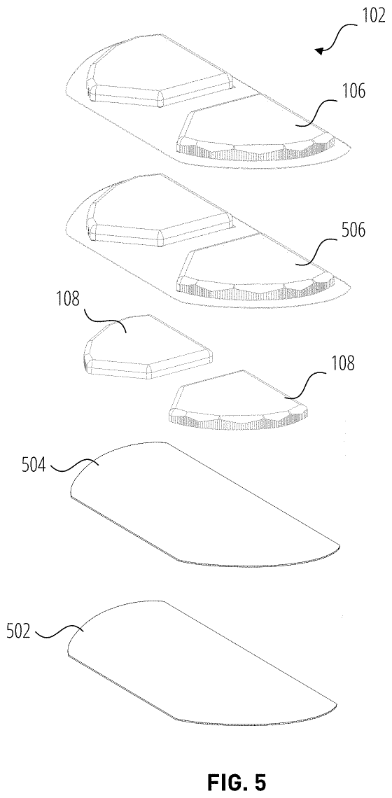

FIG. 5 is an exploded view showing the components of the EBC collector. The components of the EBC collector 102 include the thermal mass 108, a condensate forming surface 106, an attachment member 502, a support member 504, and adhesive sheets 506. The condensate forming surface can comprise a low surface energy material, such as Teflon sheet, to reduce the adhesion of the target molecule to limit target molecules from being removed from the collected EBC sample. The thermal mass is disposed between two sheets of adhesive, where one of the adhesive sheets can be thermo formed to provide a pocket for the thermal mass, and supports the low surface energy Teflon sheet comprising the condensate forming surface. The thermal mass can comprise a water and SAP polymer gel disposed between the sheets of adhesive. A collection pool can be provided for receiving the EBC, where the fluid conductor conducts the EBC from the conduction pool to the EBC testing unit. Another one of the adhesive sheets is also a support member 504 and the components of the EBC collector are configured and dimensioned so that the EBC collector can be retrofit into a pre-existing face mask. The support member can be made from other materials, such as a plastic sheet or foil. This configuration allows for the creation of a mask-based diagnostic device using many of the available face masks from a large number of world-wide manufacturers. An adhesive attachment member 502 can have a pressure sensitive adhesive side that is formulated to stick to the inside surface of the pre-existing face mask. The faces that come in contact with each other of either or both the support member and the attachment member can have surface properties that facilitate easy removal and replacement of the attachment member so that the EBC collector can be removed after use from a disposable face mask with the attachment member left on the inside surface of the face mask. The EBC collector can then be sanitized, a new attachment member added, and recycled into another mask-based diagnostic device.

An embodiment designed for scalable roll-to-roll manufacturing includes mostly sheet materials and a lost cost, safe and highly effective thermal mass. In prototype form, the sheet materials that form the EBC condensation surface, thermal mass and support member are cut to 6″×2.5″. A heat press and die is used to form a thermal mass pocket in a lamination of 0.005″ Teflon sheet (condensation surface) and double sided 3M 96042 adhesive. The 3M 96042 adhesive has a low surface energy (LSE) adhesive coated on both sides of a polyester liner, and is an example of the adhesives available that can be used for this embodiment. About 2 ml of a water/SAP gel (thermal mass) is deposited into the thermal mass pocket. This water-based thermal mass takes advantage of the high specific heat capacity of water making the chilled water-based thermal mass well suited for this application of cooling the Teflon condensation surface during a period of time needed to collect an adequate sample of EBC.

The 3M 96042 adhesive is a low surface energy adhesive that is specifically designed to stick to difficult to adhered to surface, and sticks well to the Teflon sheet and to itself, so two sheets of 3M 96042 can be used to form a water-tight enclosure for the water/SAP gel thermal mass. A second sheet of the 3M 95042 adhesive forms the support member and seals the water/SAP in the thermal mass pocket. Since the 3M 95042 remains sticky even when covered with water, a 0.003″ polyester sheet or tissue paper spacer is cut to fit within the pocket to prevent the two adhesives from sticking to each other at the location thermal mass. The collection pool is made from the same Teflon sheet material, adhered in place with the 3M 96042 adhesive to form a water-tight pool. This construction results in a very robust EBC collector that can withstand multiple sterilizations and re-uses, and is water tight and has a thermal mass pocket that is difficult to pop or tear open.

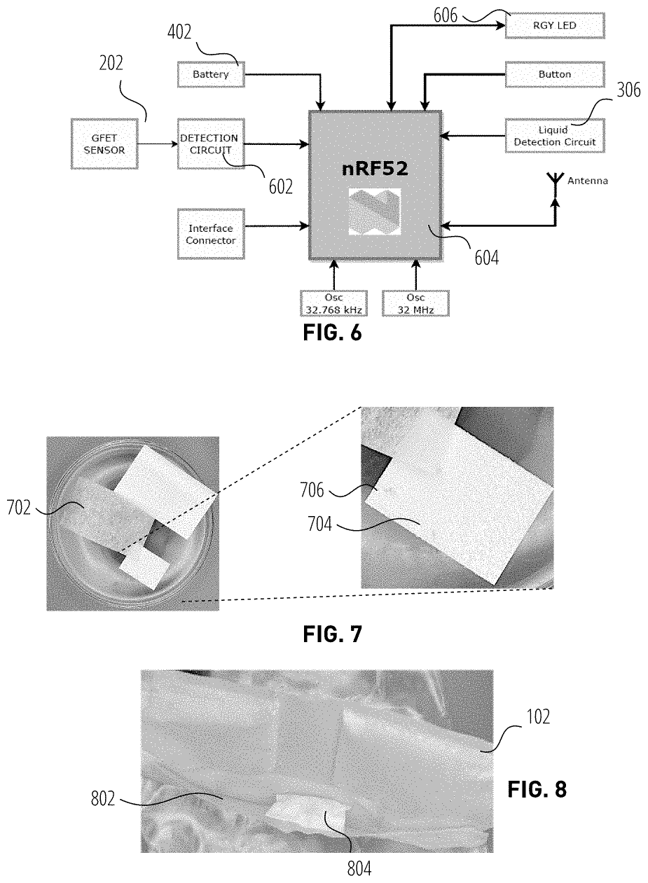

FIG. 6 is a block diagram of the electronic circuit of an EBC test unit. The EBC test unit includes a biosensor 202, a fluid detector 306, a battery 402, analysis electronics 602, communications electronics 604, and one or more test result/progress indicating LEDs 606. The analysis electronics detect the target molecule dependent on an electrical signal received from the electronic biosensor. The various components can be incorporated as elements on the printed circuit board or as components of a system-on-chip package which may also include the biosensor. The communications electronics communicates the detection of the target molecule to a user through an indicator, such as a light emitting diode or smartphone, computer or tablet display. The communications electronics can communicate the detection of the target molecule wirelessly through an optical or radio frequency (RF) signal, for example, Bluetooth communication to a smart phone or network relay device.

The fluid detector may comprise a pair of adjacent electrical conductors in a fluid detection region of the flow path. A change in conductivity between the pair is detected when the EBC sample flows in the fluid detection region. The fluid conductor comprises a microfluidics material that is selected to reduce the adhesion of the target molecule to limit target molecules from being removed from the portion of the EBC sample that flows in the flow path while the target molecule is carried along with the EBC sample to the electronic biosensor.

FIG. 7 shows components of a liquid sample concentrator with a close up view showing a SAP particulate embedded on adhesive. The liquid sample concentrator comprises a filter member 702, a SAP 704, and a SAP holding substrate 706.

FIG. 8 shows a liquid sample EBC concentrator 804 provided as a pouch adhered to a wall in a collection pool of an EBC collector. The system comprises an EBC collector 102, a collection pool 802, and an insertable EBC concentrator 804. A super absorbent polymer (SAP) and/or other water wicking/absorbing material, is provided in the flow path or in the collection pool before the EBC sample flows to the biosensor. The SAP selectively adsorbs water from the EBC sample and concentrates the target molecule in a portion of the EBC sample that reaches the electronic biosensor.

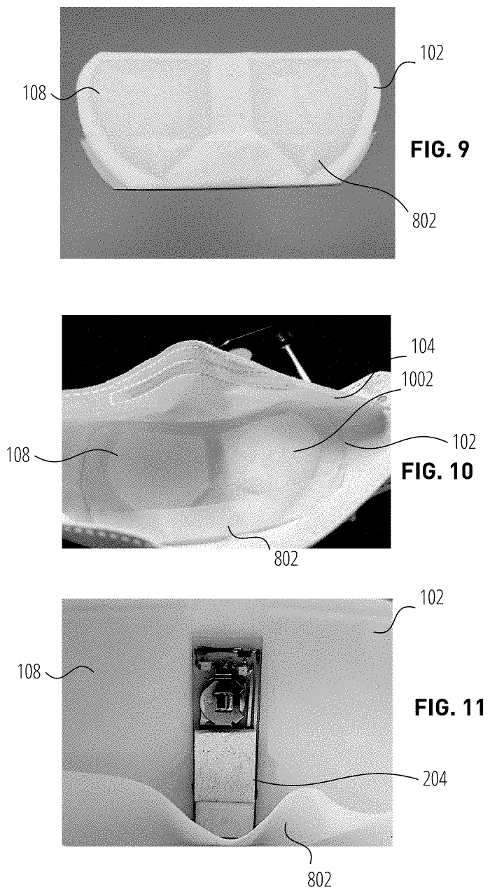

FIG. 9 shows a removable and recyclable EBC collector. The removable and recyclable components includes an EBC collector 102 with a thermal mass 108 and a collection pool 802. Depending on the application, the EBC collector can be used for sample collection alone, or for example, can be used as part of an integrated system where the EBC sample is first concentrated by a liquid sample concentrator and then the concentrated EBC sample is provided to an EBC testing unit in fluid communication with the collection pool.

FIG. 10 shows the removable and recyclable EBC collector retrofit into a pre-existing face mask. The EBC collector 102 is retrofit into an pre-existing face mask 104, and includes a thermal mass 108 and a collection pool 802. As shown, EBC droplets 1002 form during use on the condensate forming surface and can be transferred to the collection pool by the action of gravity and/or fluid transferring channels (not shown) can facilitate coalescing the EBC droplets into the EBC sample contained in the collection pool. The support member along with the other components of the EBC collector are configured and dimensioned to removably fit into a pre-existing face mask. An inside of the face mask defines a confined local environment. During use, the local environment includes breath vapor exhaled from the lungs of a user. An exhaled breath condensate collector is supported on the support member, and is disposed during use within the defined volume. A condensate forming surface converts the exhaled breath vapor into an exhaled breath condensate (EBC) sample. The EBC collector includes a thermal mass cooled before use so that during use the condensate forming surface is at a condensation forming temperature less than a confined environment temperature of the confined local environment inside of the face mask. An attachment member (shown for example in FIG. 5) removably attaches the support member and EBC collector to the inside of the face mask. The attachment member may comprise a double-sided adhesive with one side for adhering to the support member of the condensate collector and another side for adhering to the inside of the face mask.

FIG. 11 shows a removable and recyclable EBC testing unit centrally mounted on an EBC collector and in fluid communication with the collection pool. The system includes an EBC collector 102 with a thermal mass 108 and a collection pool 802, and an EBC testing unit 204 that can be provided inside the mask between the two thermal masses, or fixed to the outside of the mask with an extended microfluidics fluid conductor (not shown) accessing the EBC sample in the collection pool and transferring the sample to the biosensor on the EBC testing unit. The biosensor can be provided having a detection area for receiving the EBC sample from the fluid conductor. A collection pool can be provided for collecting and holding the EBC sample. The collection pool can also be accessible for removing the EBC sample for analysis using a table top EBC testing unit and biosensor located remotely from the mask-based diagnostic device. In addition or alternatively, the biosensor can be provided in direct fluid communication with an EBC testing unit removably integrated with the mask-based diagnostic device so that the biosensor can be refreshed and/or the EBC testing unit can be sanitized for re-use.

The removable and recyclable mask-based diagnostic device is particularly suitable for use in hospital and clinical settings, or for locations where populations of test subjects are being tested, for example at airports or arenas to screen for a communicable disease, such as the SARS-CoV-2 virus, TB or other communicable pathogen. Once a test subject has been tested, the mask based diagnostic can be collected and then mass sterilized, for example, using a UV lamp and/or a sterilizing solution containing a disinfectant. At least the components that constitute the recyclable materials are compatible with the sterilizing solution and can be first removed from a lower cost disposable face mask which can then be discarded. For example, the printed circuit board can include a conformal coating. In this case, one or more layers of conformal coatings can be applied to the PCB to make it suitable for repeated re-use and disinfection. The conformal coating can include, for example, parylene, acrylic, silicone, polyurethane and/or epoxy. In some embodiments, the electronic biosensor is held in a socket and is removable from the PCB. Tests have shown the g-FET sensor described herein can be disinfected, refreshed and reused. The electronic biosensor can be disinfected and refreshed using a reagent rinse, such as using hydrogen peroxide mixed with propylene glycol or glycerol to both disinfect and refresh the sensor so that after testing the functionalized biosensor can be reused.

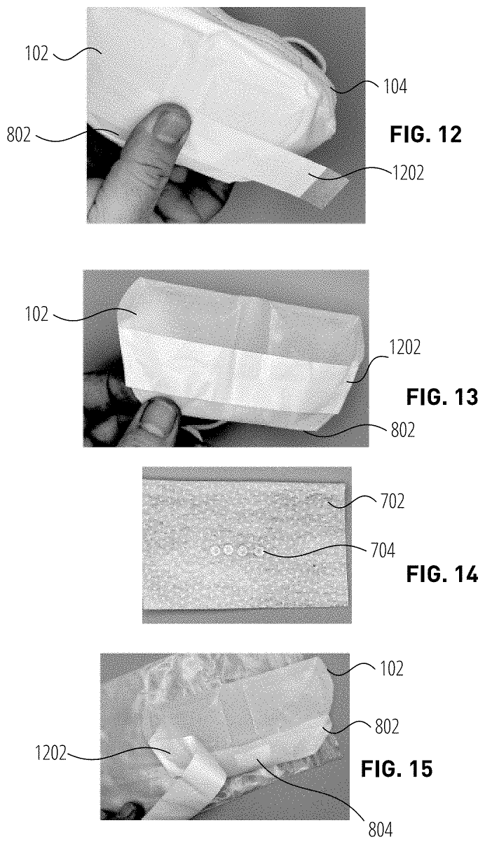

FIG. 12 shows a sealing member sealing the collection pool of a removable and recyclable EBC collector fixed to a disposable face mask. With this configuration, the sealed collection pool becomes the vessel for holding the EBC sample so the sample can be transported to a remote lab for testing, or stored as a frozen sample for later testing. The sealing member 1202 can also use the same LSE adhesive used to construct the EBC collector (e.g., 3M 96042).

FIG. 13 shows the sealed EBC collector removed from the disposable face mask. The system comprises an EBC collector 102, a collection pool 802, and a sealing member 1202. The sealing member can be, for example, a low surface energy (LSE) adhesive tape that firmly sticks to the Teflon surfaces of the condensate forming surface and the collection pool. The sealing member can be provided for sealing the EBC sample in the collection pool for transporting the EBC sample to a remote testing location.

FIG. 14 shows the components of a liquid sample concentrator. The liquid sample concentrator includes a filter member 702 and a SAP 704. The liquid sample concentrator can be used for a mask-based diagnostic sample concentrator and includes a filter member having an obverse side and a reverse side, where the filter member has a minimum particle filtering size. A super absorbent polymer (SAP) particulate is disposed on the obverse side. During use, a fluid sample comprising a carrier fluid sample containing a target molecule contacts the reverse side of the filter member. The carrier fluid sample flows along a length of the filter member where a portion of the fluid sample flows through the filter member to wet the SAP, where the super absorbent polymer selectively absorbs the carrier fluid sample to concentrate the target molecule in the carrier fluid sample flowing along the length.

FIG. 15 shows the removed EBC collector 102 with the sample concentrator disposed in the collection pool 802 being sealed by a sealing member 1202.

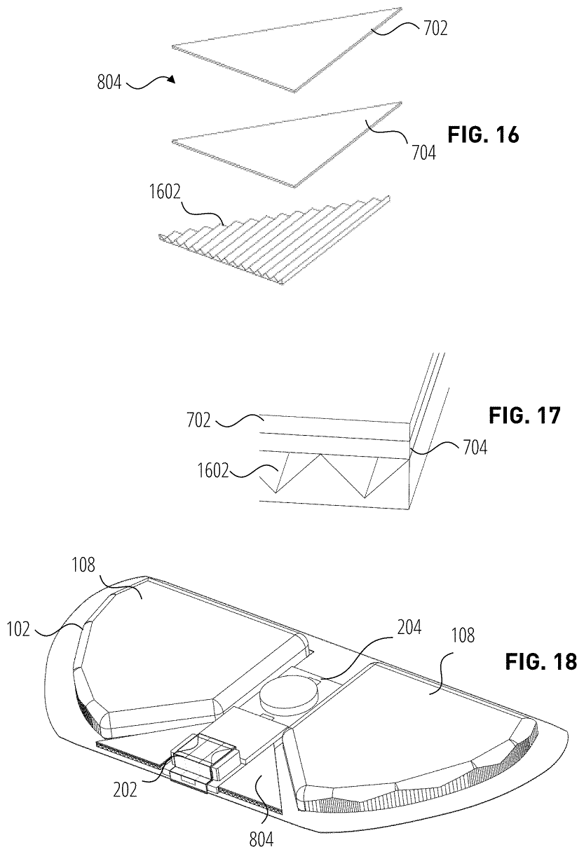

FIG. 16 illustrates a liquid sample concentrator having a low surface energy substrate, fluid conductor and selective water absorber. The components of the liquid sample concentrator includes a piece of filter paper or other suitable material that acts as a filter member 702 to pass a portion of the water from the EBC sample to a selectively absorbing material, such as SAP 704, that selectively absorbs water molecules and ions into the SAP bulk polymer and does not absorb larger molecules, such as target molecule proteins. An EBC channel 1602 may be provided to facilitate the flow of the concentrated EBC sample towards the collection pool.

FIG. 17 is a close up showing grooves of the low surface energy substrate for channeling a concentrated liquid sample to the collection pool in fluid communication with an EBC testing unit. The liquid sample concentrator includes a filter member 702, a SAP 704, an EBC concentrator 804, and an EBC channel 1602. The order and number of layers that make up the liquid sample concentrator may vary. For example, a filter layer may be adjacent to the EBC channel with the SAP layer provided over the filter layer, followed by another filter layer. Or, the filter layer may be in the form of a pouch that at least partially envelopes the SAP layer. The filter layer could be adhered to the surface of the EBC collector, with the EBC Channel and SAP layers disposed over the filter layer. The EBC channel may be obviated or formed such that it promotes the flow of the EBC towards the collection pool via capillary action.

FIG. 18 illustrates an EBC collector with a centrally mounted EBC testing unit and a pair of liquid sample concentrators. The EBC collector 102 has a thermal mass 108 and has mounted on it a biosensor 202 on an EBC testing unit 204, and an EBC concentrator 804. By this construction, as EBC droplets form on the cooled condensate forming surface, they flow due to gravity, shaking, or via fluid conducting channels (not shown) from the condensate forming surface towards the collection pool. At least a portion of these condensate droplets are received by the sample concentrator and build up at the entry point where grooves in a low surface energy substrate channel the EBC towards the collection pool. A quantity of the EBC sample contacts the fluid conductor, e.g., a filter paper sheet, and some of the EBC sample flows through the filter paper sheet and comes in contact with the SAP particulate. The SAP particulate selectively absorbs water and dissolved ions out of the EBC sample, but does not appreciably allow larger molecules or particles to be absorbed. These non-absorbed molecules and particles will flow along the filter paper as part of a concentrated EBC sample, where the target molecules become more and more concentrated as the water in the EBC sample is removed by being absorbed in the SAP particulate.

The SAP particulate can be bound to a substrate with an un-swelled size smaller than the minimum particle filtering size and a swelled size greater than the minimum particle filtering size. By this construction, during manufacturing and before use, even if the SAP particulate has a relatively smaller particulate size, and thus has more surface area and faster water absorbing properties, it remains bound up on the adhesive sheet (as shown in FIG. 7 as a composite adhesive sheet/SAP 704) so even the smaller particulate doesn't pass through the filter member. Then, during use, the SAP particulate absorbs water in the fluid sample and swells to a swelled size larger than the minimum particle filtering size.

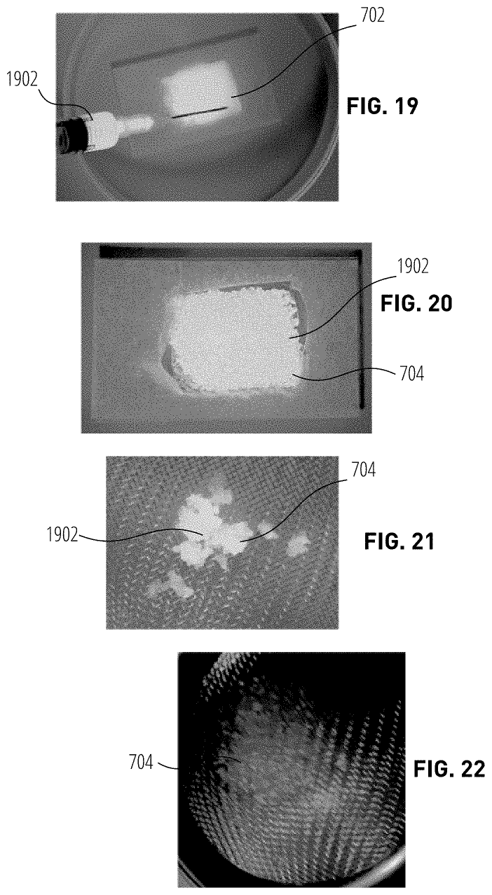

FIG. 19 shows under black light a water carrier fluid with florescent nanoparticles 1902 being disposed onto a sample concentrator having filter paper filter member 702 covering a layer of un-swelled SAP particulate embedded on an adhesive sheet. The florescent nanoparticles are 100 nm in diameter and have a similar size to a virus particle, such as the SARS-CoV-2 virus. This experiment shows that the SAP particulate is effective at absorbing water from a collected water-based sample that has a target molecule or particulate contained in the water (e.g., EBC collected from a person infected with SARS-CoV-2 virus). FIG. 20 shows swelled SAP particulate after absorbing water with florescent nanoparticles. A patch of the filter member over the swelled nanoparticles is removed so the swelled SAP glowing under black light can be clearly seen. Upon swelling at least a portion of the swelled SAP particulate may be released from being bound to the adhesive substrate but since the swelled particle size is larger than the minimum particle filtering size, it is retained at the obverse side of the filter member. The SAP particulate selectively absorbs water from the liquid sample and preferentially does not absorb the target molecule to concentrate the target molecule contained in the liquid sample.

FIG. 20 shows swelled SAP particulate after absorbing water with florescent nanoparticles. The 100 nm florescent nanoparticles in water are about the same size and in the about the same carrier medium as virus particles in and EBC sample, and are useful to demonstrate the effectiveness of using SAP to selectively absorb water and concentrate a target molecule or particulate.

FIG. 21 shows an un-rinsed portion of the swelled SAP particulate with florescent nanoparticles. The SAP particulate glows under the black light due to the florescent nanoparticles that cling to the surface of the swollen particulate. However, rinsing the swell particulate removes the florescent nanoparticles leaving the swollen SAP particulate clear and not glowing under the black light.

FIG. 22 shows the portion of swelled SAP particulate after rinsing away the florescent nanoparticles. The experiment with florescent nanoparticles shows that virus sized particles (e.g., SARS-CoV-2 virus particles) are too large to be absorbed into the expanding polymer chains of a super absorbent polymer that is being swelled by the absorption of water. Since EBC is nearly all water, the use of SAP will effectively concentrate the target molecules (e.g., virus particles) contained in the tested EBC sample.

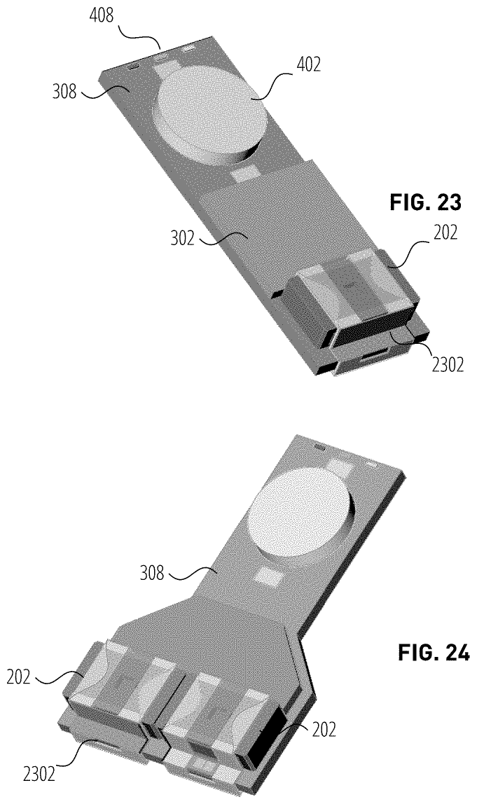

FIG. 23 illustrates the EBC testing unit showing a dissolvable surfactant film disposed in the fluid flow path before the sample flow reaches the electronic biosensor. This embodiment of the EBC testing unit includes a biosensor 202, a wick 302, a printed circuit board 308, a battery 402, an LED 408, and also a surfactant film 2302. The EBC fluid sample includes analytes or target biomarkers in a mostly-water fluid carrier. The dissolvable surfactant film is placed in the fluid flow path before the sample flow reaches the electronic biosensor so that, for example, a lipid viral shell of the SARS-CoV-2 virus can be breached through the chemical action of the surfactant on the lipids making up the viral shell. This causing lysing of the virus to release internal biomarkers, such as the virus N-protein, which can then be detected using a biosensor that is functionalized with capture molecules specific to binding to the N-protein.

FIG. 24 illustrates a version of the EBC testing unit showing two parallel biosensors for simultaneously receiving the fluid sample at the detection area of each biosensor. In many use-cases it is advantageous to test for two or more different biomarkers in one tested sample. For example, a person infected with SARS-CoV-2 can be tested for both the S- or spike protein of the virus. These S-proteins are typically found on the outside of the virus particle and can be detected by a biosensor that is functionalized with a capture molecule designed to specifically bind to this target biomarker, the S-protein. In parallel, or sequentially, the EBC sample can be directed to flow to a second biosensor as well. In this case, prior to reaching the second biosensor the EBC sample can be treated with a lysing chemical (e.g., contained in a dissolvable surfactant film) to release a second detectable biomarker, such as the virus N-protein. The second biosensor is functionalized with the capture molecules that are designed specifically to bind to this second biomarker, the N-protein.

It may be advantageous to allow the EBC or liquid sample to flow over the detection area, allow flow volume and time to elapse so that the target molecules bind to the capture molecules, and then before taking a test reading of the electrical signal generated by the biosensor, rinse the detection area of the biosensor with a clean solvent, such as de-ionized water. The rinse will remove from the detection area at least some of the potentially test-confounding molecules and ions that may be present in the EBC, including surfactant, lysing materials and other non-target molecules and ions. The target molecules that bind with the immobilized capture molecules will be held at the detection area. The rinse can be done intermittent with the application to portions of the test sample to the detection area, so that confounding molecules can be rinsed away giving additional target molecules more opportunity bind with the immobilized capture molecules and change the electrical characteristics of the biosensor.

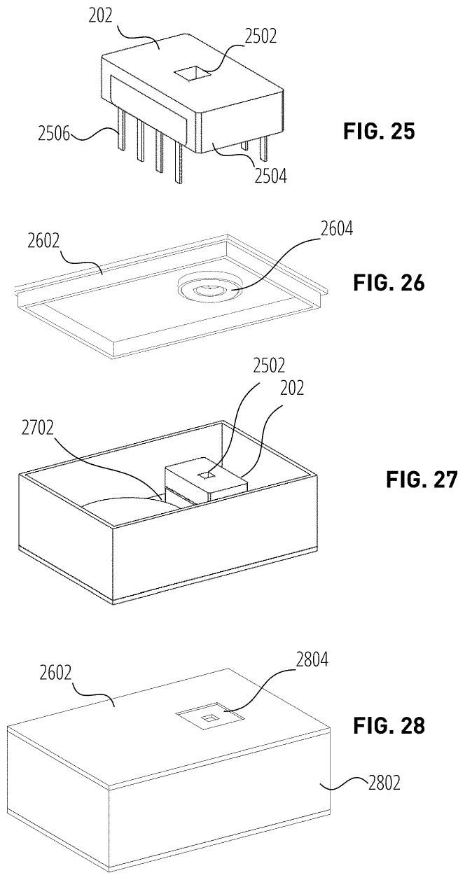

FIG. 25 is an isolated view of a semiconductor DIP packaged biosensor for incorporation in a printed circuit board. The semiconductor DIP package comprises a biosensor 202, a detection well 2502, an encapsulant 2504, and pins 2506. Although g-FET biosensors have been known for several years, they have generally been used by researchers in a laboratory setting.

The functioning of the g-FET biosensor requires access to a portion of the top surface of the semiconductor device. At the top surface the detection area is provided with the charge transport layer of graphene, and on the graphene layer capture molecules are immobilized that are designed to bind to the target biomarker molecules and thereby cause a detectable change in the electrical output of the g-FET. However, this necessary feature of leaving a portion of the top surface of the semiconductor device exposed is unusual and requires a different semiconductor package than is usually available for packaged semiconductor electronic devices. As a result, g-FET biosensors are typically sold as bare die components with connecting pads for connecting the g-FET features (e.g., source, drain and gate) to typical research lab equipment, such as a probe station. To make a g-FET biosensor scalable for mass production, a solution is needed to package the g-FET bare die so that it can be conveniently connected with a printed circuit board. In accordance with another non-limiting embodiment, a semiconductor DIP packaged biosensor is provided where the conventional Double In-line package, DIP, configuration is used to make the biosensor easy to handle and easy to place into a printed circuit board. There are other semiconductor package types that can be used in accordance with the embodiment shown in FIG. 25, including, for example, SOP/SOIC/SO (Small Outline Package), QFP (Quad Flat Package), QFN/LCC (Quad Flat Non-leaded Package, BGA (Ball Grid Array Package) and CSP (Chip Scale Package). In all cases, access to a portion of the top surface of the semiconductor g-FET device is provided through a window or well built into a top portion of the packaging encapsulating material, which could be formed in place with the window or well, or provided as a pre-formed lid having the window or well. This detection well in the semiconductor packaged electronic devices obtains a configuration of the electronic biosensor that can be easily handled by existing electronic circuit production equipment at scale.

As described herein, embodiment of the mask-based diagnostic device can include an EBC testing unit including a PCB with packaged electronic biosensor that are mounted directly onto the EBC collector. This allows a self-contained system that is well suited for massive deployment for at-home testing and other-the-counter sales. However, the collection of the EBC sample and the testing of the collected sample can be done remote from each other. For example, the EBC collector can be used to obtain an EBC sample, and then the sample sealed within the collection pool as shown in FIGS. 12-13. The accumulated EBC sample can also be removed from the collection pool using a pipette or dropper, placed in a vial and sealed. In these cases, the EBC sample can be transported to a remote lab for analysis. As another alternative, the collected EBC sample can be obtained immediately after the mask-based diagnostic device is used, and then the sample placed into the well of a table top EBC testing unit.

FIG. 26 illustrates the lid 2602 with an o-ring 2604 for use in a table top EBC testing unit.

FIG. 27 illustrates the housing in which sits an EBC testing unit PCB comprising a biosensor 202, a detection well 2502, and a socket 2702.

FIG. 28 illustrates an assembled table top EBC testing unit showing a testing well for reaching a liquid sample. The table top EBC testing unit is housed in a housing 2802, and includes a lid 2602 with a testing well 2804. In this case, the test subject does not have to wait for the EBC sample to be transported to a remote lab, wait for the lab to perform the analysis of the EBC, and then wait even longer to get the test results either directly from the lab or relayed through a health care provider. The table top EBC testing unit can be used at the same location where the EBC sample is obtained, and is suitable for clinical or at-home use.

Also, the table top EBC testing unit can be used for detecting biomarkers and target molecules in other fluids. For example, blood, urine, saliva, sweat, interstitial fluid, tears, mucus, gastrointestinal lavage, or other bodily fluid. Also, the small size and portability, along with the wireless smartphone APP-based user interface and network relay communications capabilities as described herein can be used for environmental testing of waste water, potable water, HVAC condensate and room-scale environmental and bio-sensing.

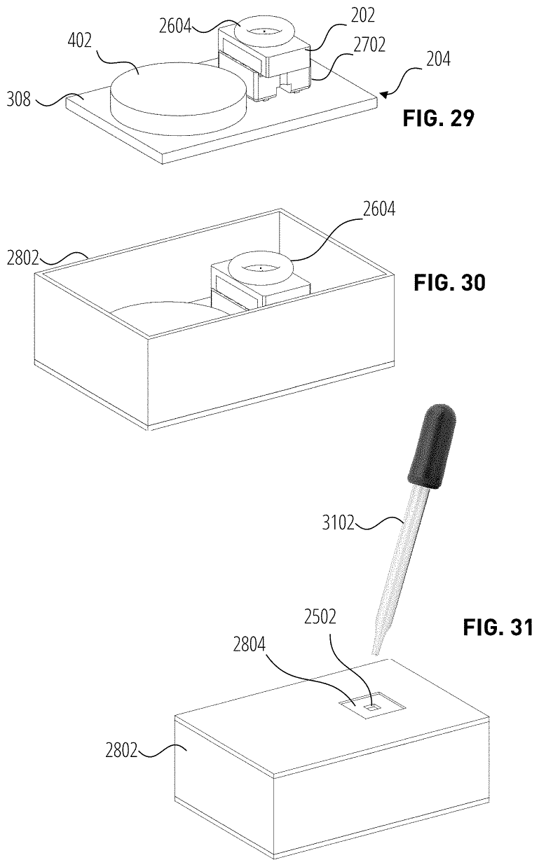

FIG. 29 illustrates a PCB board with a coin cell battery and DIP socket for receiving the semiconductor DIP packaged biosensor. The PCB board includes a biosensor 202, mounted in a socket 2702 to connect with the electronics of a printed circuit board 308, with a battery 402.

FIG. 30 illustrates the PCB board sitting in the housing with an o-ring 2604 for sealing the detection well of the electronic biosensor to the lid of the table top EBC testing unit.

FIG. 31 illustrates the table top version of the EBC test unit showing a dropper 3102 for disposing a liquid sample into a testing well 2804 in the lid. As shown, an o-ring or other gasket member can be used to seal around the test well in the lid of the table top housing and the detection well of the biosensor. The dimensions of the test well, o-ring and detection well can be selected so that a suitable quantity of the EBC sample remains contained and stagnant during the operation of the EBC testing electronics. In this case, the EBC sample does not flow over the detection well, but instead a quantity of the EBC sample remains in place in the test well/o-ring/detection well volume and a duration of time can be allowed for the target molecules in the EBC sample to bind to the capture molecules immobilized on the graphene layer of the biosensor.

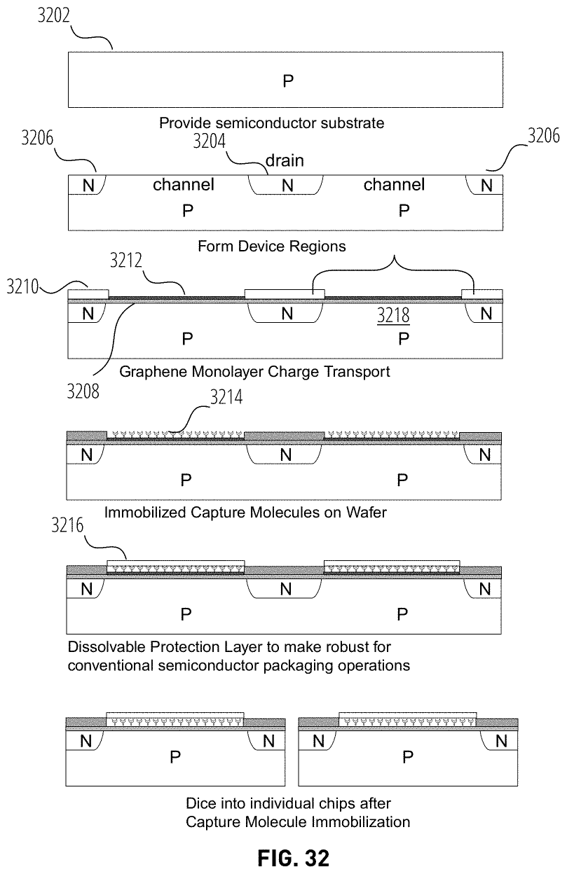

FIG. 32 illustrates the steps of making a semiconductor wafer of graphene field effect transistor biosensors with a protection layer patterned in place over the detection interfaces of the g-FET biosensors. The semiconductor substrate wafer 3202 is processed into discrete bare die semiconductor devices using primarily known semiconductor manufacturing techniques and equipment. The formed bare die is a field effect transistor (FET) that includes a drain 3204 and a source 3206 that form a channel, and an insulator layer 3208, with electrodes 3210 formed for connection with an external circuit. Unique to this exemplary embodiment and not found in conventional semiconductor wafer of FETs is a charge transfer layer 3212 with immobilized capture molecules 3214, and a protection layer 3216.

In accordance with a non-limiting embodiment, a device structure and a method for making a semiconductor biosensor comprises the steps of providing a semiconductor substrate wafer of one conductivity type. The semiconductor substrate can be P- or N-type conductivity. A plurality of semiconductor device regions 3218 are formed in the semiconductor substrate wafer. Each semiconductor device region comprising at least a source region and a drain region defining there between a channel region of the one conductivity type with the source region and the drain region of an opposite conductivity type. As shown, the substrate is P-type, and the source and drain regions are N-type. A detection area is formed over the channel region. The detection area including a charge transfer layer, such as a monolayer of graphene, hexagonal boron nitride (h-BN), silicene, germanium, black phosphorus (BP), transition metal sulfides, etc. At the wafer level, capture molecules are immobilized on the charge transfer layer of at least a portion of the plurality of semiconductor device regions. A protection layer can optionally be provided as a pattern over the immobilized capture molecules or over the entire surface of the wafer after the immobilization step. By performing the immobilization step at the wafer level, the numerous advantages of the mature manufacturing process of semiconductor fabrication can be utilized to create a highly scalable manufacturing process for creating a functionalized biosensor. The alternative of performing the immobilization step after dicing the wafer is typically what is being performed now, and far from an optimized mass production method and mostly useful for small scale and research purposes. In accordance with this non-limiting exemplary embodiment, as shown at the bottom of FIG. 32, the individual semiconductor die or devices are separated from the semiconductor substrate wafer, where each device includes at least one semiconductor device region and where the step of separating is performed after the step immobilizing. Typically, the functionalization of the biosensor or the immobilizing of the capture molecules onto the charge transport layer is done by first depositing a solution of a linker molecule in a carrier, such as de-ionized water. After incubation at a predetermined temperature and time, an end of the linker molecule binds to the molecules making up the charge transfer layer. For example, pyrene can be used as the linker molecule that has one end of the pyrene molecule bonded to the carbon lattice of a graphene charge transfer layer layer.

After the linker molecule is immobilized, in another incubation step a capture molecule that is designed to bind to a specific target molecule is immobilized on the charge transport layer by attaching the capture molecule to the other end of the linker molecule. For example, the pyrene linker molecule can be used to attach an antibody, aptamer, nanoCLAMP, nano-body, or other capture molecule on the graphene charge transport layer to form a functionalized graphene field effect transistor (g-FET) biosensor. Following the processing steps where immobilization of the capture molecules is done at the wafer level allows the efficiency and precision of relatively small devices to be handled and processed simultaneously, so that, for example, one functionalization process can result in a very large number of functionalized bare die devices. Also, immobilizing may include providing the capture molecules as polarized capture molecule conjugates including a linker molecule end and capture molecule end, and applying an electrostatic field to orient and drive the linker molecule end toward the charge transfer layer to facilitate binding the linker molecule end with the charge transfer layer.

As shown in FIG. 32, a protection layer can be patterned over just the detection areas of each device region to protect the relatively delicate immobilized capture molecule/graphene charge transport layer. The protection layer can also be formed over the entire surface of the wafer. The protection layer can be dissolvable in a solvent, such as water, or other polar or non-polar solvent, depending on the processing steps and use-case of the functionalized biosensor. By providing this protection layer at the wafer level, before dicing or otherwise separating the wafer into individual semiconductor devices, the exemplary embodiment continues to make use of the efficiencies and precision of semiconductor manufacturing machines and techniques, with the end result being a bare die device that is more easily handled by conventional semiconductor packaging equipment and processes such as pick and place, wire bonding and encapsulation. The protection layer can be patterned at the wafer level, for example, so that the protection layer is only located over the detection interface. The detection interface can be functionalized at the wafer level, so that, for example, the protection layer protects the delicate graphene charge transport layer and immobilized capture molecules. The detection interface can be functionalized after dicing the wafer, so the protection layer protects the graphene charge transport layer and the protection layer is rinsed away prior to functionalization (e.g., where functionalization is done at the packaged device level). In either case, the patterned protection layer can leave the contact pads of the g-FET exposed so that the steps of wire bonding and packaging of the bare die can be performed without having to remove the protection layer.

The optimized protection layer can include materials and process steps selected to create a removable protection layer that can be peeled, dissolved, etched, evaporated, etc., once the protective nature of the layer is no longer desired. For example, a multilayered structure may include desiccants or oxygen getters. The materials can include composite structures with particulate and matrix configurations, or a multilayered torturous path structure to create a barrier to oxygen, moisture or other environmental factors that could reduce the shelf life or usefulness of the functionalized biosensor, and protect against damage caused by material processing and handling steps that take the bare die from wafer through to the in-service device. The protection layer can be configured as a multilayered structure so that a more robust surface film is provided that can be removed during a processing step of the bare die from wafer to in-service device. Then, with the more robust film removed, during the use of the biosensor, the sample material, such as EBC, is able to rinse away the remaining protective layer so the capture molecules are able to bind with the target molecules. For example, a water-soluble adhesive film which may be made from sucrose can provided to protect the immobilized capture molecules from oxygen and moisture, and a removable polymer sheet film can be adhered to the adhesive film to provide more robust protection during pick and place, wire bonding and encapsulation, shipping and handling steps.

As an example, the performance of the immobilized capture molecule may be susceptible to degradation by a spatial change in orientation or distance relative to the charge transfer layer. To lock in the position of the immobilized capture molecule a sugar and water solution can be deposited onto the surface of the wafer to at least cover the detection area of the device regions. The water is evaporated leaving a dry sugar layer protecting the immobilized capture molecules. Over the sugar layer, a more robust film can be adhered (e.g., a thin cellophane) or formed from solution (e.g., poly(methyl methacrylate). During at least some of the processing steps from wafer to in-service device, the more robust film can remain in place, which is removed at some point before use leaving the sucrose layer to be dissolved and rinsed away by water deposited from a dropper just prior to use, or from the EBC sample flow during use.

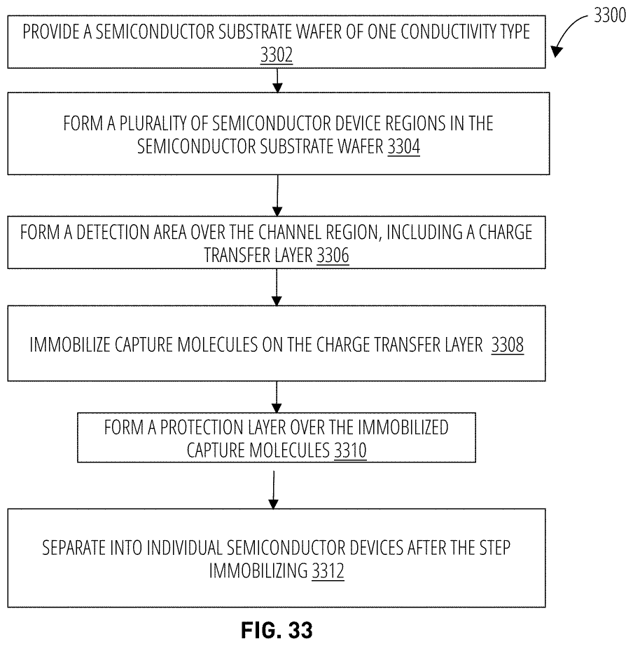

FIG. 33 is a flow chart (3300) for making a semiconductor wafer of graphene field effect transistor biosensors with capture molecules immobilized on the detection interface prior to dicing the semiconductor wafer. In block 3302, a semiconductor substrate wafer of one conductivity type is provided. In block 3304, a plurality of semiconductor device regions are formed in the semiconductor substrate wafer, each semiconductor device region comprising at least a source region and a drain region defining there between a channel region of the one conductivity type with the source region and the drain region of an opposite conductivity type. In block 3306, a detection area is formed over the channel region, the detection area including a charge transfer layer. In block 3308, capture molecules are immobilized on the charge transfer layer of at least a portion of the plurality of semiconductor device regions. A protection layer can be formed to protect the charge transfer layer and immobilized capture molecules, block 3310. In block 3312 the individual semiconductor devices from the semiconductor substrate wafer are separated with each separate bare die comprising at least one semiconductor device region. In accordance with this embodiment, the step of separating the bare die from the wafer is performed after the step immobilizing. All of the steps are capable of being performed using mostly typical semiconductor processes and equipment. By keeping the individual semiconductor devices all ganged together with the precision and compactness of the semiconductor wafer, a large number of functionalized bare die semiconductor sensors can be formed efficiently. The addition of the protection layer over the immobilized capture molecules allows the resulting bare die to be more easily processed for pick and place, wire bonding and packaging operations without damaging the relatively delicate capture molecules and charge transfer layers. The immobilization of the capture molecules can also be performed after packaging or dicing, with the protection layer formed to protect the charge transfer layer until the protection layer is rinsed away or otherwise removed to expose the charge transfer layer for linker and capture molecule functionalization.

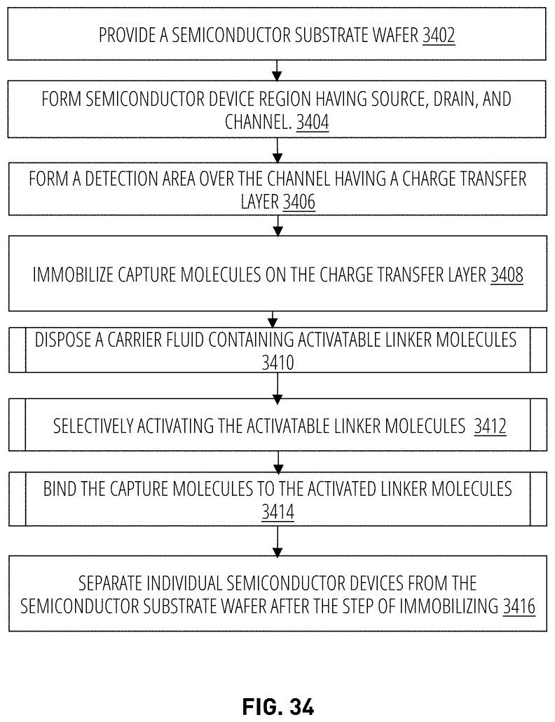

FIG. 34 is a flow chart for making the semiconductor wafer of graphene field effect transistor biosensors with capture molecules immobilized onto selectively activatable linker molecules. In block 3402, a semiconductor substrate wafer of one conductivity type is provided. In block 3404, a plurality of semiconductor device regions are formed in the semiconductor substrate wafer, each semiconductor device region comprising at least a source region and a drain region defining there between a channel region of the one conductivity type with the source region and the drain region of an opposite conductivity type. In block 3406, a detection area is formed over the channel region, the detection area including a charge transfer layer. In block 3408, capture molecules are immobilized on the charge transfer layer of at least a portion of the plurality of semiconductor device regions.

To selectively immobilize different capture molecules on different g-FET devices, activatable linker molecules disposed in a linker carrier fluid is deposited over the surface of the semiconductor wafer. (block 3410). Prior to activation, the activatable linker molecules do not bind to the free-floating capture molecules. In block 3412, the activatable linker molecules are selectively activated to form activated linker molecules immobilized at some of the charge transfer layers, the activated linker molecules bind with the free-floating capture molecules. The activatable linker molecules are photo-activated, for example, where the linker molecules include a photo-sensitive end that becomes active for binding with capture molecules after being irradiated with a pre-determined wavelength of light or invisible radiation.

In block 3414, the capture molecules are bonded to the activated linker molecules. Then, in block 3416, individual semiconductor devices are separated from the semiconductor substrate wafer, each comprising at least one semiconductor device region, wherein the step of separating is performed after the steps of immobilizing the capture molecules.

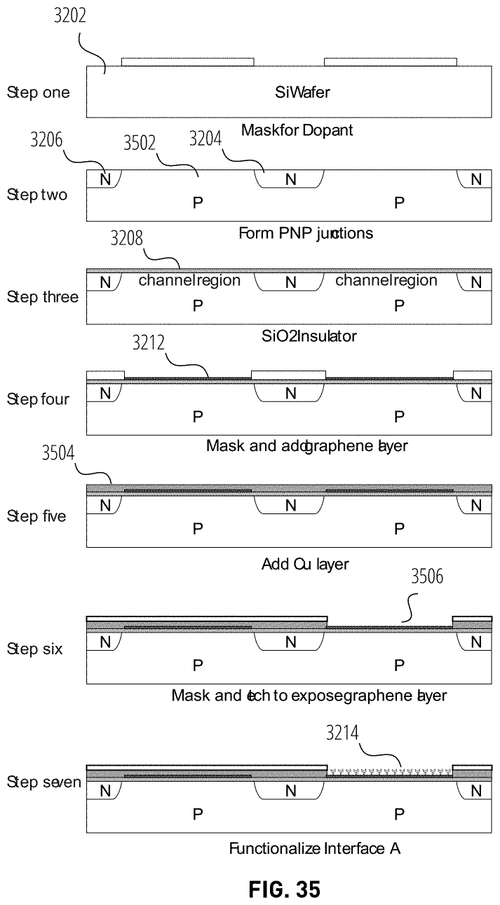

FIG. 35 illustrates the steps for making the semiconductor wafer of graphene field effect transistor biosensors with selectively immobilizing a second type of capture molecules without disturbing the capture molecules previously immobilized on the first type of functionalized biosensors, and with a dissolvable protection layer formed over the surface of the wafer. The process comprises the formation on a semiconductor substrate wafer 3202 various features that create a g-FET sensor that includes a source 3206, an insulator layer 3208, capture molecules 3214, a channel 3502, a drain 3204, a charge transfer layer 3212, and a copper layer 3504. An Interface A 3506 is selectively functionalized before being protected, and then an Interface B is exposed for functionalization so that Interface A and Interface B can be formed adjacent to each other on the semiconductor wafer, and functionalized with different capture molecules. For example, Interface A can be functionalized to detect a Flu virus biomarker in an EBC sample, and Interface B can be functionalized to detect a SARs virus biomarker in the same EBC sample. By this process, a semiconductor bare die can be created that can have two or more g-FETs each functionalized to detect different biomarkers.

For example, adjacent g-FET bare die structures, such as those available as from Graphenea, Spain, can be functionalized at the wafer level and used with the same EBC collector, electronics, microfluidics, etc., of a mask-based diagnostic device that can detect and distinguish FluA, FluB and SARs biomarkers present in the EBC collected from a test subject. Although a non-limiting exemplary embodiment is described, different steps and materials can be used in the processes described herein to form the resulting singulated bare die semiconductor devices having the characteristics shown that enable the efficiencies, installed manufacturing capacity and mature technologies of conventional semiconductor processes and materials to form a semiconductor packaged and functionalized biosensor. The singulated bare die devices can have one or more differently functionalized g-FET devices, or the same capture molecules can be functionalized on one ore more g-FETs on a bare die or multiple dies, or any combination, depending on the application and target biomarker(s).

In accordance with a non-limiting embodiment, a method for making a semiconductor biosensor includes providing a semiconductor substrate wafer of one conductivity type. In the example shown, a Si wafer is doped as a P-type substrate, but could be doped as an N-type substrate. A mask if provided (step one) and a plurality of semiconductor device regions are formed in the semiconductor substrate wafer by forming N-type regions so that each semiconductor device region incudes at least a source region and a drain region (step two). A channel region of the one conductivity type is defined between the source region and the drain region of an opposite conductivity type. A thin SiO2 insulator may be formed over the wafer surface (step 3) or patterned so the SiO2 is formed only over the channel region. A detection area is formed over the channel region and includes a charge transfer layer, such as graphene (step four). The graphene can be formed through a mask or otherwise patterned so that a graphene layer is only formed over the channel region.

An etch-able layer can be formed over the surface of the wafer to cover the graphene and other exposed surface area of the wafer (step five). For example, Cu can be deposited over the wafer surface. In the case where more than one type of capture molecule is to be immobilized at the detection area of specific device regions, a resist layer is patterned so that the Cu layer can be selectively removed to expose only the graphene layers that are to be functionalized (step six). Capture molecules are then immobilized on the charge transfer layer of at least a portion of the plurality of semiconductor device regions indicated as Interface A (step seven).

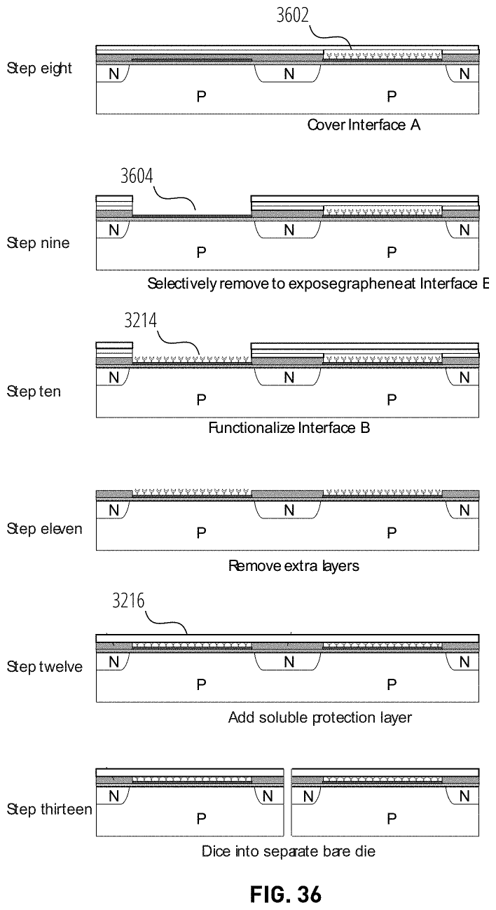

FIG. 36 illustrates the steps for making the semiconductor wafer of graphene field effect transistor biosensors with selectively immobilizing a second type of capture molecules without disturbing the capture molecules previously immobilized on the first type of functionalized biosensors, with a dissolvable protection layer formed over the surface of the wafer. The semiconductor substrate wafer 3202 is processed using conventional techniques, including using a mask 3602 to protect functionalized Interface A while exposing and immobilizing a different type of capture molecules 3214 on Interface B 3604. A protection layer 3216 is formed after the final capture molecule immobilization step.

In this non-limiting exemplary embodiment shown in FIGS. 35 and 36, the step of immobilizing the capture molecules may comprise immobilizing a first type of capture molecule on the charge transfer layer of each of a first sub-set of the plurality of semiconductor device regions and immobilizing a second type of capture molecule on the charge transfer layer of each of a second sub-set of the plurality of semiconductor device regions. Also, a protection layer pattern can be formed over at least the charge transfer layer of said each of the first sub-set prior to immobilizing the second type of capture molecule.

At least two different types of capture molecules can be immobilized on corresponding charge transport layers of respective device regions. Interface A has been functionalized by immobilizing a first type of capture molecules onto the graphene layer, while Interface B has been covered. Interface A is then covered by a removable layer to protect Interface A and prevent a second type of capture molecule from becoming immobilized on Interface A (step eight). This allows for a simple dip or spin coating, or other liquid deposition techniques to be used to selectively immobilize the first type of capture molecules only at exposed graphene layers. For example, the entire surface of the wafer can be flooded with a linker in carrier fluid (e.g., pyrene in de-ionized water) and incubated to bind the pyrene molecule to the exposed graphene. A rinse water and spin process removes any remaining linker/carrier off the wafer surface. This linker/carrier and rinse water that spins off the wafer can be collected for concentration and reuse. A similar second spin coat, incubation, rinse step is then performed to bind the first type of capture molecule to the pyrene linker and selectively functionalize the first type of capture molecule only onto the exposed graphene layer at Interface A and then in later processing steps, the spin coat, incubate, rinse process used again to immobilize a second type of capture molecule only onto the exposed graphene layer at Interface B.

The removable layer that covers Interface A is compatible with the immobilized first type of capture molecules on Interface A, so that the graphene and immobilized capture molecules are protected and covered, but can later be exposed without device-limiting damage done to the graphene and immobilized capture molecules. For example, the removable layer can be comprised of a water soluble material that can be later dissolved away without damaging the functionalized biosensor constituents. Interface A can be covered by the removable layer by masking, or direct deposition, such as jetting, or micro deposition using a digital dispenser so that the removable layer is only formed onto selected areas of the wafer surface to cover Interface A and leave the graphene layer at Interface B is exposed. Alternatively, or additionally, a mask 3602 can be selectively patterned to leave functionalized Interface A covered and expose Interface B for functionalization.

Another exemplary process for this selective patterning and exposing can be done by first forming a removable layer over the entire surface of the wafer substrate (step eight). A patterned layer is then formed (e.g., using Cu as the patternable layer and masking then etching). After the pattern is formed in the patternable layer, the removable layer is removed only from the graphene layer at Interface B (or removed everywhere except for the functionalized Interface A) (step nine). A second type of capture molecule is immobilized at Interface B (step ten). The extra layers are removed from the surface of the wafer so that the separately functionalized Interface A and Interface B and the source and drain connection pads are exposed (step eleven). Additionally, a protection layer can be formed over functionalized Interface B so that both Interface A and Interface B are protected when the extra layers are removed to expose the connection pads for the sources and drains. To make the semiconductor devices more robust for further processing using conventional semiconductor manufacturing machines and techniques, a soluble protection layer can be formed at least over Interface A and Interface B (step twelve). As shown, the soluble protection layer is formed over the entire wafer surface. However, the soluble protection layer can be formed in a pattern so that the source and drain connection pads are exposed. The wafer is then diced so that separate bare die graphene field effect transistor semiconductor devices are formed (step thirteen).

These bare die devices are now ready for the next steps for semiconductor electronic circuit component manufacturing, such as, testing, wire bonding, packaging, and then insertion as a discrete electronic component of a printed circuit board, such as the EBC testing unit PCB described herein. In accordance with this embodiment, when the individual semiconductor devices are separated from the semiconductor substrate wafer, each comprises at least one semiconductor device region. Since the step of separating the individual semiconductor devices is performed after the step immobilizing the capture molecules, the semiconductor devices are processed all the way through to a functionalized bare die biosensor bare die while remaining in the high precision and compact arrangement of a semiconductor wafer. Also, as described, a dissolvable protection layer can also be formed over the surface of the wafer to make the resulting diced bare die semiconductor devices suitable for packaging into packaged semiconductor electronic circuit devices using conventional manufacturing techniques and equipment. Further, an insulator layer can be formed over the channel region prior to forming the detection area, and additionally or alternatively, a dielectric layer may be formed over the channel region prior to forming the detection area. The gate stack capacitance influences many of the important performance parameters of g-FET. For example, the dielectric layer may be a a Hf- or Ln-based gate stack can be formed to improve the permittivity. The charge transfer layer can comprise a graphene layer, or other charge transport material, and the semiconductor device structure can be formed as a field effect transistor on a doped semiconductor substrate. Alternatively, other semiconductor device configurations are possible, and the detection area, separately functionalized Interfaces A, B, etc, can be formed on a different kind of substrate, such as glass (with lower costs but without the features available when formed on a semiconductor substrate). A protection layer can be also formed over the charge transfer layer after the step of immobilizing the capture molecules and before the step of separating the individual devices. In any case, the protection layer can be formed over the charge transfer layer to protect the immobilized capture molecules and is removable by a solvent or other process after the step of separating, with the protection layer left in place throughout the handling and further processing steps, to protect from device destroying damage to the immobilized capture molecules on the charge transfer layer.

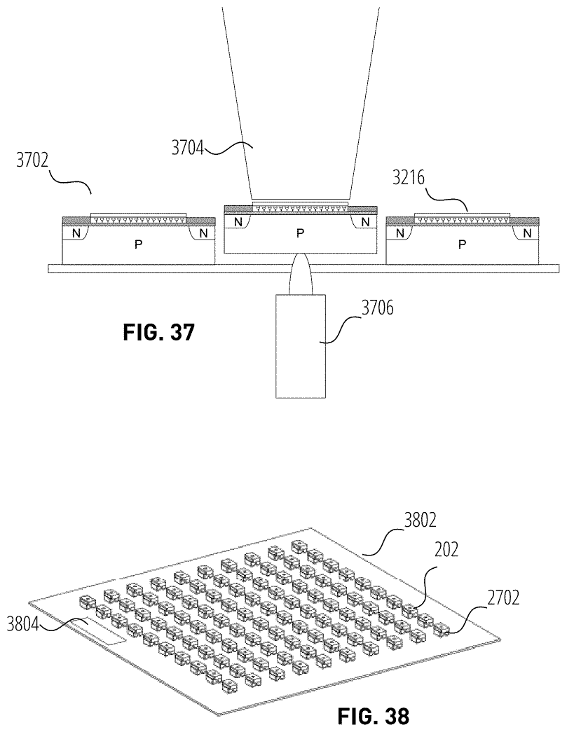

FIG. 37 illustrates a diced semiconductor wafer of g-FET biosensors with an individual g-FET biosensorbare die 3702 with a protection layer 3216, being picked by a nozzle 3704 and ejector 3706 of a conventional pick and place machine. The semiconductor devices are held together on a wafer and have been processed all the way through to being a functionalized bare die biosensor with a dissolvable protection layer formed at least over the immobilized capture molecules and charge transport layer so that packaged semiconductor electronic circuit devices can be formed using conventional manufacturing techniques and equipment.

FIG. 38 illustrates a work holder 3802 for holding a plurality of semiconductor packaged g-FET biosensors for batch functionalization using an automatic dispensing system. The work holder 3802 holds a plurality of biosensors 202 that are placed in sockets 2702 and may also be electrically connected with a common test circuit 3804. A plurality of DIP packaged electronic biosensors can be held in corresponding DIP sockets provided on a work holder. As an example, in the case of unfunctionalized packaged biosensors, a batch functionalization process can be done using precision digital depending equipment for dispensing a linker and capture molecule in carrier fluid to immobilize the capture molecule onto the charge transport layer. The work holder can be provided as a printed circuit board, with an onboard test circuit used to monitor changing in electrical characteristics of the individual biosensors before, during and/or after the functionalization steps.

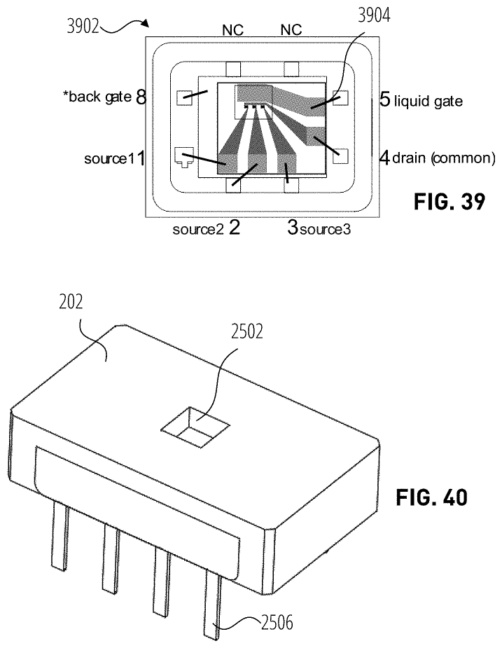

FIG. 39 illustrates a wire bonding process for packaging a g-FET biosensor bare die in a conventional DIP package 3902 with wire bonds 3904 connecting the bare die pads with pins of the DIP package 3902. In the wire bond example shown, sources (source1-source3) of g-FET semiconductor devices are connected to pins 1-3, respectively, of a DIP semiconductor package lead frame. A common drain is wire bonded to pin 4, a liquid gate is connected to pin 5 and the metalized die bonding area is bonded to pin 8. A conductive adhesive or solder is used to bond the back of the semiconductor device to the metalized die bonding area so that pin 8 provides and electrical connection to a back gate of the g-FET biosensors. The liquid gate has an exposed conductive surface at the detection area, and at least the g-FET semiconductor device charge transport layers are also exposed in the detection area.

FIG. 40 illustrates a semiconductor DIP packaged g-FET biosensor 202 with a detection well 2502 and connection pins 2506 for providing a liquid sample to the detection interface of the g-FET. In accordance with a non-limiting exemplary embodiment, a bare die sensor is provided with connection pads. The bare die sensor has at least one charge transfer layer. The transfer layer may be left unfunctionalized, and can have a dissolvable protective layer formed over it (for example a water-soluble thin film formed over the graphene if unfunctionalized, or formed over the graphene with immobilized capture molecules if functionalized). The transfer layers of two or more g-FETs (which could be formed adjacently with each other, have different sub-configurations, such as different combinations of parallel and serial connected g-FETs sharing common conductive and semi-conductive features). The g-FETs are functionalized with at least one type of immobilized capture molecules protected by a removable protection layer. The bare die sensor is placed into a packaged die frame having pins for connecting the sensor to an external circuit.

As an example, the bare die can be a g-FET biosensor purchased from a foundry, such as Graphenea located in Spain. The connection pads are connected to the pins, for example, using a conventional wire bonding process. The wire bonded bare die sensor is encapsulated in a housing or in a encapsulating material, where the encapsulation or housing is formed having a through hole located in the region of the charge transfer layer to leave during use the immobilized capture molecules exposed. The encapsulation material can be selected so that the through hole can be provided and so that a strong organic solvent, such as DMF (Dimethylformamide) can be used in a functionalization process performed after encapsulation. Typical encapsulation materials, such as an epoxy, may not be suitable for this unique packaged semiconductor application because of the need for forming a through hole to access the detection area on the bare die, and because of the use of the strong organic solvent for the functionalization process. In accordance with a non-limiting exemplary embodiment, a silicone sealant that has little or no solubility in DMF can be used to protect the wire bonds and other features and provide for the through hole to access the detection area. A cap with a through hole can be adhered to the top of the silicone sealant to provide a hardened top surface of the packaged device to facilitate electronic circuit processes, such as pick and place operations, and to define a consistent z-axis height for a batch functionalization process and for mounting the EBC testing unit in a housing with an o-ring seal, as shown, for example, in FIGS. 29-31.

The protection layer can be removed by dissolving or other removable technique so that during use, the immobilized capture molecules are exposed for receiving a sample containing a target molecule that binds to the capture molecules and causes a detectable change in electrical characteristics of the sensor. When the detection well receives a liquid sample, the pins of the packaged semiconductor biosensor can be used to test for a change in electrical characteristics between the various constituents of the semiconductor biosensor. For example, the source/drain current can be measured, liquid gate/drain current, back gate/drain current, etc. For example, when the biosensor is operated as a g-FET, the source/drain current will depend on a change in current caused by the target molecules being captured by the immobilized capture molecules on graphene charge transport layer.

The semiconductor packaged device can be soldered directly onto a printed circuitboard, or held in a socket, resulting in an electronic circuit element that can be easily handled by conventional circuit board manufacturing equipment, materials and processes. The device packaging can have one or more functionalized or unfunctionalized semiconductor biosensors that can be protected by a dissolvable protective layer. Depending on the application for use, the type of capture molecule immobilized, type of charge transport layer, etc., the protective layer can be dissolvable in a polar or non-polar solvent, can be meltable, freeze-to-release, etc. so that manufacturing processing steps, storage and shipping needs can be accommodated and/or requirements for clinical, at-home, point-of-care, etc., use can be performed. For example, a water dissolvable protection layer may need to be rinsed away before functionalizing (e.g., at the wafer level the protection layer is applied before immobilizing capture molecules) or before use (e.g., at-home diagnostic use where a mother rinses the detection well before placing the diagnostic on her child). The dissolvable protection layer can be applied after functionalization at the packaged device level, or printed circuitboard level, making the sensor highly adaptable for a wide range of biological or environmental testing, for a wide range of diseases, health conditions and ambient environmental conditions.

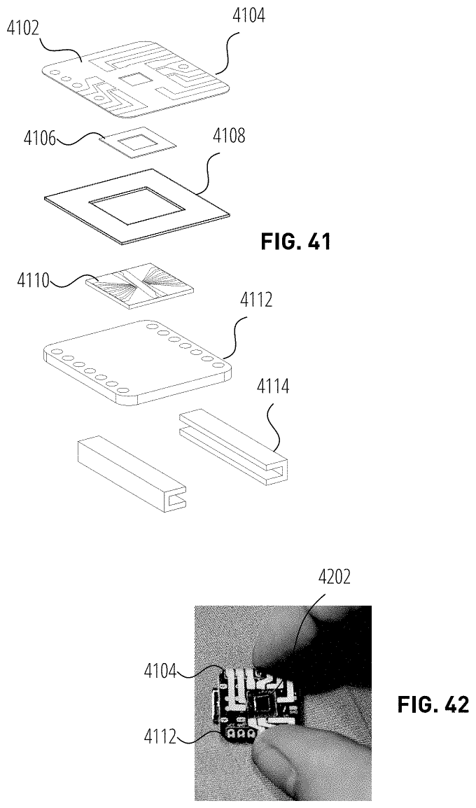

FIG. 41 is an exploded view illustrating a non-wire bonded alternative construction for connecting a bare die g-FET biosensor to a microprocessor PCB. The system comprises a flex substrate 4102, a lead pattern of printed silver ink 4104, a silicone gasket 4106, z-axis conductive tape 4108, a g-FET 4110, a microprocessor PCB 4112, and a retainer 4114.

FIG. 42 shows a prototype non-wire bonded alternative construction of a bare die g-FET biosensor connected to a microprocessor PCB 4112 with a detection well 4202 formed in the flex substrate for receiving a liquid sample. In this non-limiting, exemplary embodiment, a connection substrate, such as a plastic, glass, rigid or flex circuit substrate, has etched copper or printed conductive ink leads formed on a surface, with a through hole forming a detection well. A silicone, o-ring or other gasket can be provided for sealing the detection well to the detection area of a bare die g-FET device. A z-axis conductive adhesive or tape is used to connect the conductive leads formed on the connection substrate and to connect connection points of a microprocessor/RF communication, or other electronic circuit PCB. This configuration avoids the conventional wire bonding, encapsulation and other process steps and materials used for connecting a bare die electronic device to a printed circuit board.

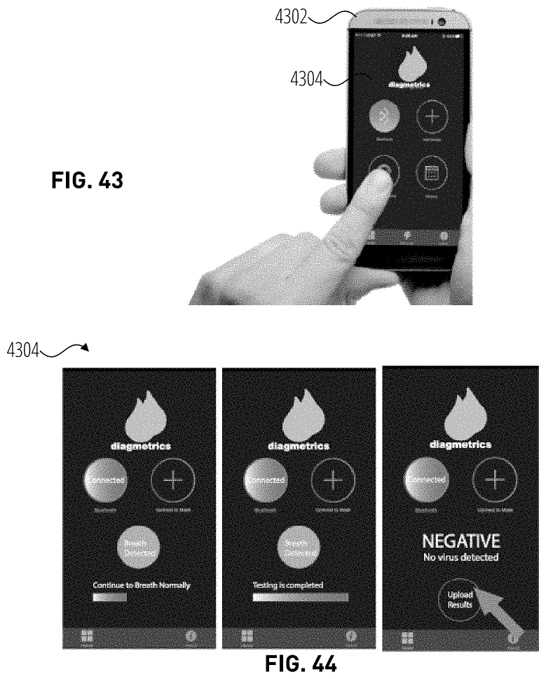

FIG. 43 shows a smartphone 4302 having an APP 4304 for communicating with the EBC testing unit and conveying information to the phone user and/or to a remote network server.

FIG. 44 shows screens of a smartphone APP providing a graphical user interface for conveying the steps for conducting a test using a mask-based diagnostic device and receiving information indicating the test results.

The EBC testing unit described herein can be configured with bluetooth communication processor or microcontroller. The electronic biosensor produces a direct-to-electrical signal output (that is, there is no visual or optical test result, since the biosensor detects a target molecule through a mechanism that results directly into a change of electrical output detectable at the connection leads to the sensor constituents (e.g., source, drain, gate leads). This direct-to-electrical signal is particularly suite for low cost digital communication and signal processing. The signal from the biosensor can be sent as a raw value, which is then analyzed, for example, using the processing power of a cellphone or network server, or the signal from the biosensor can be processed by a microcontroller or processor provided in direct electrical communication with the biosensor, such as contained on the EBC testing unit PCB. The processing of the signal can be split among devices, with wired or wireless signal transmission among the devices. For example, a smartphone and APP can be used as shown to indicate instructions for use and a final test result to the user or healthcare provider administering the test to a patient. The mask-based diagnostic device is particularly useful as an at-home or point-of-care test, where a test subject only has to open the APP on their smartphone, put on the mask and breathe. Such a simple test system can change the course of disease outbreaks, such as viral pandemics, and be used to proactively monitor an at-risk patient for many diseases and health conditions that are detectable from biomarkers contained in blood, lungs, airways and exhaled breath of the test subject. Also, the mask-based diagnostic device is particularly suitable for aggregating data obtained from large populations regionally located or scattered throughout the world by using existing network infrastructures, such as cellular networks and the Internet, to obtains copious data useful, for example, by Artificial Intelligence (AI) and/or Machine Learning (ML) algorithms.

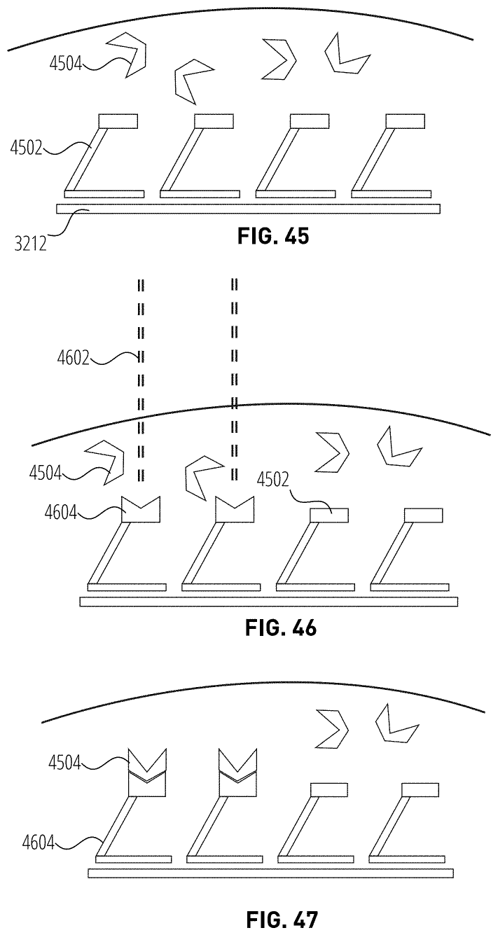

FIG. 45 illustrates activatable linker molecules before activation, with free-floating capture molecules in a liquid carrier. An unactivated linker 4502 is bonded on a charge transfer layer 3212 of a sensor, and covered with a carrier fluid containing free-floating capture molecule1 4504. Prior to activation, the unactivated linker is not receptive to binding with the capture molecule1.

FIG. 46 illustrates some activatable linker being selectively activated by photo radiation for binding to the capture molecules. A pattern of radiation 4602 selectively activates the activated linker. For example, a laser pulse can be focused to selectively irradiate only areas where the unactivated linker 4502 is converted to unactivated linker 4502 that are receptive to binding to capture molecule1 4504. The radiation 4602 can be patterned, by x/y scanning and pulsing a laser beam, or patterned through a mask, or flooded onto the entire surface to activate the linkers all at once.

FIG. 47 illustrates the activated linker binding with free-floating capture molecules. The capture molecule1 4504 binds with an unactivated linker 4502 to selectively immobilize the capture molecules on a change transfer layer to form a functionalized g-FET sensor.

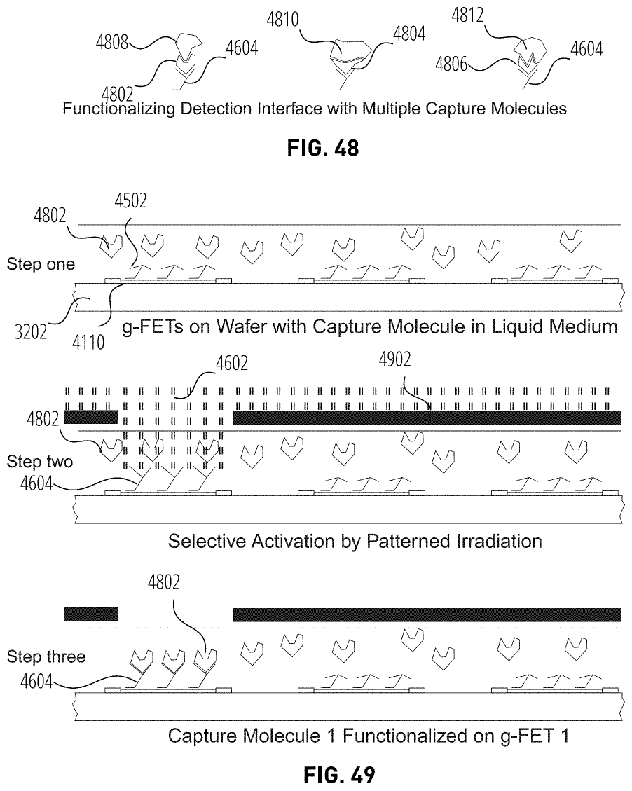

FIG. 48 illustrates different capture molecules for detecting different capture molecules and bond to activatable linkers. In this embodiment, an unactivated linker 4502 is selectively activate by patterned radiation to bind different capture molecules to functionalize different g-FETs formed on a semiconductor wafer. The capture molecules include two or more different types (e.g., capture molecule1 4802, capture molecule2 4804, and capture molecule3 4806. Each capture molecule selectively binds with a different target molecule (respectively, target molecule1 4808, target molecule2 4810, target molecule3 4812). The detection interface of different g-FET devices on the same wafer can be functionalized with a distinct capture molecule that can be used to detect the presence of a class of biomarkers. For example, in the case of SARS corona viruses, the N-protein within the viral envelope of these virus is usually preserved from variant to variant, while the S-protein will mutate and become a variant or sub-variant of a predecessor variant or original viral strain. The N-protein biomarker can then be considered a class of biomarker that is detectable for many different strains of SARS.

The same detection interface of a semiconductor sensor, or more detection interfaces of adjacent sensors, can be functionalized with multiple capture molecules. For example, if the same detection interface has multiple capture molecules immobilized on the same charge transfer layer, a screening test can be used where a detected change in signal output caused by capturing at least one type of biomarker indicates a potential health condition.

As a specific but non-limiting example, researchers have identified these six different biomarkers that indicate a potential risk of pancreatic cancer: ApoA1, CA125, CA19-9, CEA, ApoA2, and TTR (see, Kim H, Kang K N, Shin Y S, et al. Biomarker Panel for the Diagnosis of Pancreatic Ductal Adenocarcinoma. Cancers (Basel). 2020; 12(6):1443. Published 2020 Jun. 1. doi:10.3390/cancers12061443). One or more electronic biosensor interface can be functionalized with two or more types of capture molecules that selectively bind to a respective one of these six identified biomarkers. Gastro-intestinal lavage obtained during a colonoscopy can be used as a biosample that is tested. A screening test for pancreatic testing can use a change in the signal output from the biosensor that exceeds a predetermined threshold as an indication that the test subject may have pancreatic cancer and should be tested further. Similarly, the detection interface of six adjacent semiconductor sensors can be functionalized with a respective capture molecule that selectively binds with one corresponding biomarker. Probabilistic analysis of the signal out from each sensor can be used to determine if the test subject should undergo additional testing for pancreatic cancer.

FIG. 49 illustrates the steps of selectively binding a first sub-set of capture molecules to activatable linker molecules. A plurality of g-FETs 4110 are formed on the semiconductor substrate wafer 3202, each g-FET having device regions formed as described herein. Each device region forms a g-FET and includes a source, a drain and at least one channel region. At least one of an insulator layer and a dielectric layer is formed over each channel region and a detection area including a charge transfer layer is formed over said at least one of an insulator layer and a dielectric layer.

For the semiconductor construction of an optimized sensor, the main g-FET sensor performance parameters include: the drain current, ID, the trans-conductance, gm, the channel conductance, gD, the threshold voltage, VT, the gate stack reliability, and the gate direct tunneling current density, JDT. Most of these parameters are influenced by the gate dielectric capacitance, Cdi, channel mobility, lch, metal-semiconductor work function difference, /MS, gate stack charge density, Qgsc, interface trap density, Dit, and bulk dielectric trap density, Dbt, cf. (see, High Permittivity Gate Dielectric Materials, Samares Kar, DOI 10.1007/978-3-642-36535-5)

The gate stack capacitance influences many important aspects of the g-FET sensor. To improve the permittivity at the charge transfer layer, and the biosensor performance characteristics such as sensitivity, the insulator/dielectric layer can be a multi-layered stack with a high-k material core, including at least one of HfO2, La2O3, HfSiO, HfAlO, HfNO, HfSiON, ErTiO5, SrTiO3, LaScO3, LaAlO3, GdScO3, LaLuO3, La2Hf2O7, Gd2O3, La2SiO5, SrHfO3.

An unactivated linker 4502 is incubated and immobilized on the charge transfer layers of the g-FETs. Capture molecules are selectively immobilized on the charge transfer layers of the g-FETs. Different types of capture molecules can be immobilized on the different g-FETs using selective photo-activation of the unactivated linker molecules. The activatable linker molecules are first immobilized on the charge transfer layers through an incubation step as described herein. A capture molecule carrier fluid containing the capture molecules as free-floating capture molecules is disposed over a top surface of the semiconductor substrate wafer covering the plurality of device regions. For example, spin and/or dip coating can be used to form a thin film of the carrier fluid as a liquid medium containing a first type of free-floating capture molecules (capture molecule1 4802) on the surface of the wafer (step one). Prior to activation the activatable linker molecules are relatively less receptive to binding to the free-floating capture molecules. In practice, it is important that the chemistry that causes the capture molecule to bind to the linker molecule can be initiated by a selective process, such as patterned photo-radiation 4602. For example, the photo-radiation can be flooded towards the surface of the wafer and a shadow mask 4902 used to selectively activate the activatable linker molecules to form activated linkers 4604 immobilized at some of the charge transfer layers (step two). The activated linkers 4604 are receptive to binding with the free-floating capture molecules while the unactivated linkers 4502 do not bind with the capture molecules. Once the pattern of activated linker molecules is formed, incubation at an appropriate time and temperature is used to bind the capture molecules selectively to the activated linker molecules (step three).

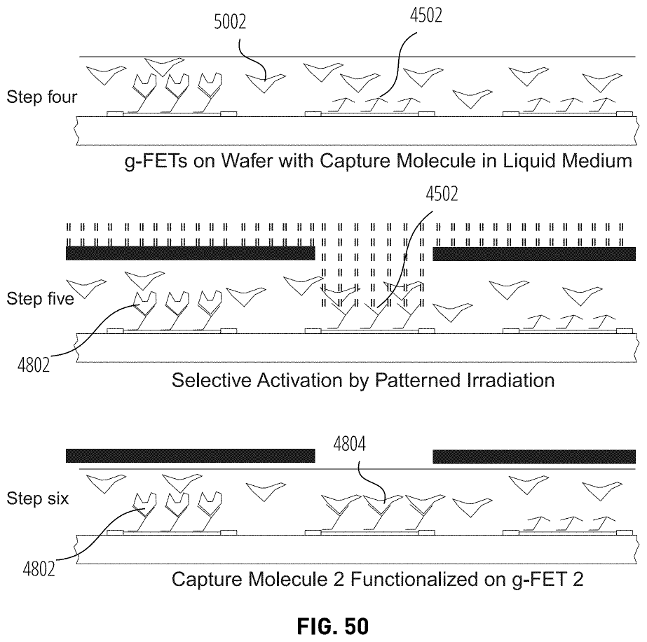

FIG. 50 illustrates the steps of selectively binding a second sub-set of capture molecules to activatable linker molecules. After the first type of capture molecules (capture molecule1 4802) is immobilized on a first sub-set of the g-FETs formed on the semiconductor wafer, the wafer contains a first sub-set of g-FETs that are functionalized with the first type of capture molecules (capture molecule1 4802) along with unfunctionalized g-FETs that have unactivated linker 4502 immobilized on the charge transfer layers. The wafer can be rinsed and spin coated with a carrier fluid containing a second type of free-floating capture molecules (capture molecule2 5002) on the surface of the wafer (step four). The selective photo-radiation through a shadow mask, or selective laser patterning, can be then be used to selectively activate the linker molecules at a second sub-set of the g-FETs (step five). After an incubation step, the second sub-set of g-FETs is functionalized with the second type of capture molecules (capture molecule2 5002) (step six). This process can be completed any number of times so that the semiconductor wafer has a variety of g-FETs functionalized for detecting different target molecules. For example, capture molecule1 can be designed to capture a FluA biomarker, capture molecule2 can be designed to capture a FluB biomarker, and capture molecule3 (not shown) can be designed to capture a SARs virus biomarker. The three g-FETs functionalized to capture the three different biomarkers (FluA/FluB/SARS) can be formed adjacent to each other so that when the wafer is diced, this grouping of g-FETs remain together on a single bare die semiconductor biosensor. This ganged multiple biomarker sensor can be packaged as a single discrete electronic circuit device or otherwise connected to the electronics of an EBC testing unit as described herein. During use, for example, for detecting FluA/FluB/SARS in EBC, the EBC sample is received at a detection area that contains the three differently functionalized g-FETs, and an output of each g-FET can be sampled and scanned in sequence over time by the electronics of the EBC testing unit.

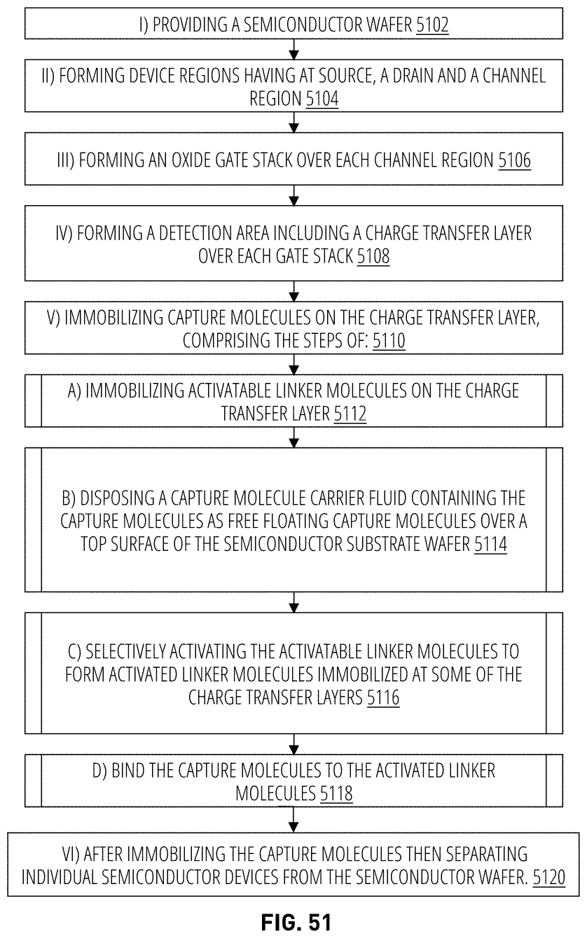

As shown in flowchart FIG. 51, the steps for forming functionalized bare die sensors through selective linker activation includes providing a semiconductor wafer (block 5102). Device regions are formed in the semiconductor wafer, each comprising a source, a drain and at least one channel region (block 5104). A gate oxide stack is formed comprising at least one oxide that has a lower dielectric constant, K, with relatively less ability to store electrical energy and is more of an insulator layer and at least one oxide that has a higher K and is more of a dielectric layer with relatively greater ability to store electric energy in an electrical field. The gate oxide stack is formed over each channel region (block 5106). In block 5108, a detection area is formed in the device regions to create unfunctionalized g-FET sensor semiconductor structures. These detection areas each include a charge transfer layer over the at least one of an insulator layer and a dielectric layer. Capture molecules are immobilized on the charge transfer layer (block 5110). The immobilization of the capture molecules includes first immobilizing activatable linker molecules on the charge transfer layers (block 5112). A capture molecule carrier fluid containing the capture molecules as free-floating capture molecules is disposed over a top surface of the semiconductor substrate wafer covering the plurality of device regions (block 5114). Prior to activation, the activatable linker molecules are relatively less receptive to binding to the free-floating capture molecules. The activatable linker molecules are selectively activated to form activated linker molecules immobilized at some of the charge transfer layers (block 5116). Some of the g-FET devices on the wafer have detection areas with activated linkers that are ready to be functionalized, and others have detection areas that are not going to be functionalized until the linker molecules immobilized on their charge transfer layers are activated. The activated linker molecules are bind with the free-floating capture molecules. The capture molecules in the carrier fluid bind to the activated linker molecules (block 5118). If additional capture molecules are to be functionalized on the other g-FET, as described herein, the steps of selectively activating the activatable linker molecules and binding the capture molecules to the activated linkers can be performed for one more additional types of capture molecules. Once all the g-FETs that are to be functionalized while on the wafer have been functionalized and the different types (or at least one type) of capture molecules have been immobilized on the g-FETs while still all ganged together on the wafer, individual semiconductor devices having functionalized g-FETs are separated from the wafer (block 5120).

An additional step of providing a protection layer over the functionalized and/or unfunctionalized g-FETs can also be performed either prior to or after separating the individual semiconductor devices from the wafer. The activated linker molecules can be provided as free-floating molecules in a carrier liquid that is flooded over the surface of the semiconductor wafer, and the selective activation can be perform while the the free-floating unactivated linker molecules in the carrier fluid after an incubation step to immobilize the unactivated linker onto all of the charge transport layers. Alternatively, the selective activation can be performed after the immobilization of the linker, after removing excess linker and carrier fluid, and before or after the capture molecule carrier fluid and capture molecules is disposed over the semiconductor surface.

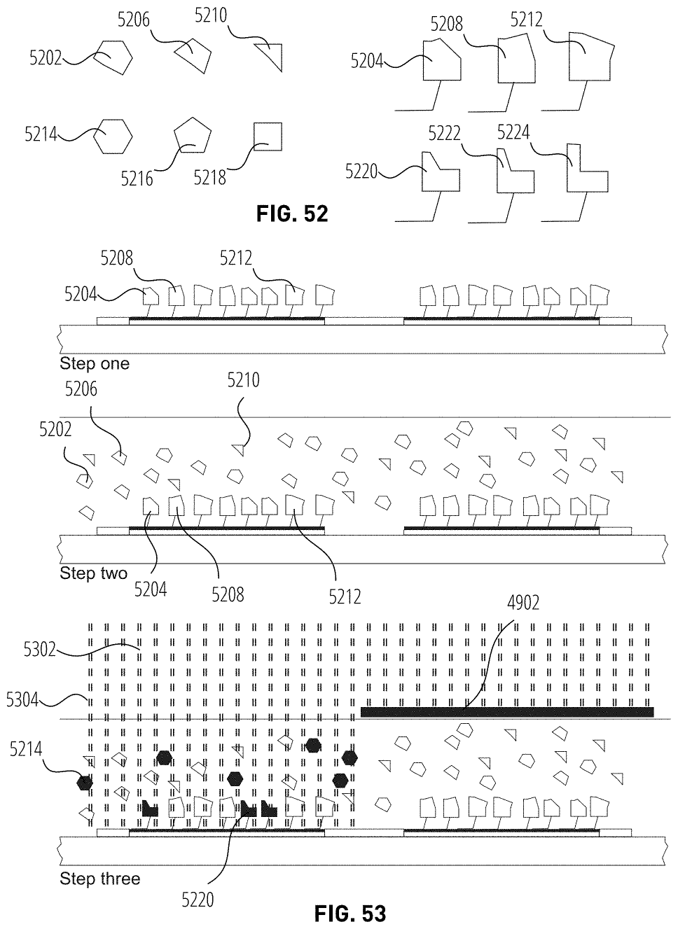

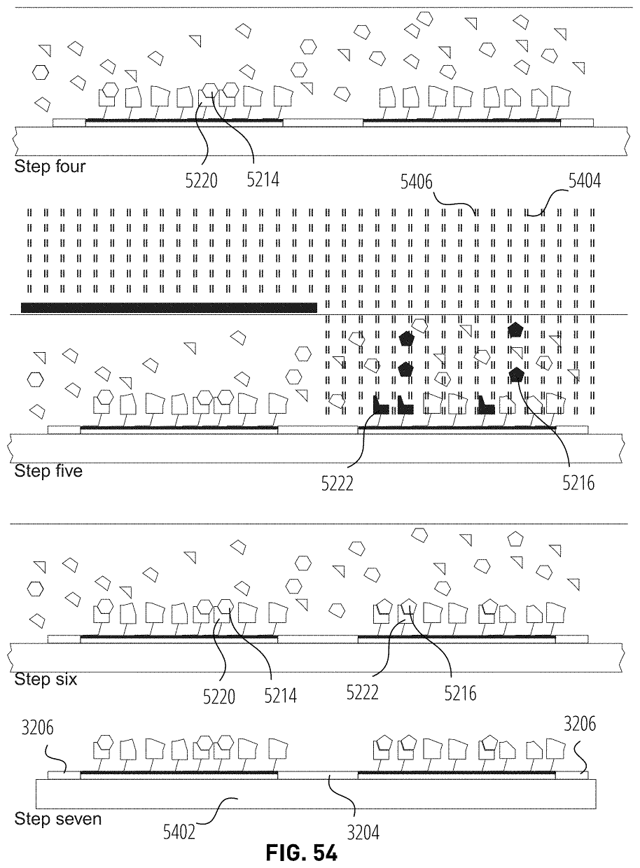

FIG. 52 illustrates activatable capture molecules, activated capture molecules, activatable linker molecules and activated linker molecules used for wafer-level functionalization of semiconductor biosensors. The activatable and activated molecules includes a first set of activatable capture molecules 5202, a first set of activatable linker molecules 5204, a second set of activatable capture molecules 5206, a second set of activatable linker molecules 5208, a Nth set of activatable capture molecules 5210, an Nth set of activatable linker molecules 5212, a first set of activated capture molecules 5214, a second set of activated capture molecules 5216, an Nth set of activated capture molecules 5218, a first set of activated linker molecules 5220, a second set of activated linker molecules 5222, and an Nth set of activated linker molecules 5224. As described herein, these molecules are disposed to form two or more g-FET sensors that are separately functionalized at the wafer-level for detecting different target molecules. A bare die sensor is constructed with two or more separately functionalized g-FET biosensors. The bare die is fabricated using the efficiencies and scalable manufacturing inherent with wafer-level processing.

Researchers has explored various photo-initiated chemical processes, such as photoclick chemistry, where chemical reactions are initiated by selective irradiation of light. See, for examples, Marcon, et al. Photochemical Immobilization of Proteins and Peptides on Benzophenone-Terminated Boron-Doped Diamond Surfaces, Langmuir, 2010, 26(2), 1075-1082; Hensarling, et al., “Clicking” Polymer Brushes with Thiol-yne Chemistry: Indoors and Out, J. Am. Chem. Soc, 2009, 131, 14673-14675; Fairbanks, et al., Photoclick Chemistry: A Bright Idea, Chemical Reviews, 2021, 125, 6915-6990.

In accordance with a non-limiting, exemplary embodiment, photo-initiated molecular binding is utilized to selectively immobilize different types of capture molecules on the charge transfer layer of different g-FET devices.

FIG. 53 illustrates the steps for selectively binding a first set of activated capture molecules to a first set of activated linker molecules.

In accordance with a non-limiting exemplary embodiment, a semiconductor wafer is provided with device regions formed. Each device region includes a source, a drain and at least one channel region. A gate oxide layer over each channel region and a detection area including a charge transfer layer is formed over the gate oxide layer. Different types of capture molecules are selectively immobilized on the charge transfer layer of different device regions. In this embodiment, first set of activatable linker molecules 5204, second set of activatable linker molecules 5208 through to a Nth set of activatable linker molecules 5212 are all immobilized on all of the charge transfer layers of all of the device regions. As shown, a first set of activatable linker molecules 5204, a second set of activatable linker molecules 5208 and a Nth set of activatable linker molecules 5212 are first immobilized on the charge transfer layer of device regions (step one). The number of sets of activatable linker modules that are immobilized will general correspond to a number of different biomarkers in the multiple biomarker sensor bare dies 5402 ultimately formed (see, step seven in FIG. 54). Each respective set of the activatable linker molecules is activated for binding by a different corresponding wavelength of linker-activating radiation. That is, when irradiated with the correct wavelength or band of wavelengths, a photo-chemical change at a binding end of the linker molecule makes the molecule receptive to binding with a corresponding capture molecule that is also photo-activated. One or more of the activated linkers can be made receptive to binding and one or more of the activated capture molecules can also be made receptive to binding. By patterning the applied radiation, selected features, such as selected device regions of a plurality of device regions on the semiconductor wafer can be functionalized with a desired capture molecule or more than one capture molecule.

As an example, a screening diagnostic test for pancreatic cancer can be made using a g-FET biosensor that has a single charge transport layer that is functionalized with two or more known biomarkers that indicate potential pancreatic cancer. For example, capture molecules designed to bind with one or more of the biomarkers ApoA1, CA125, CA19-9, CEA, ApoA2, and TTR can be functionalized onto the charge transport layer. A body fluid sample that may contain the biomarkers can be gastro-intestinal lavage obtained during a colonoscopy. Since multiple capture molecules that will selectively bind with corresponding biomarkers are functionalized onto a single charge transfer layer, the degree of change in the test signal can be taken as a screening test indication for the target disease. Additionally, or alternatively, each charge transfer layer of two or more adjacent g-FET devices on the wafer can be separately functionalized with a different capture molecule, then packaged in a packaged biosensor with multiple g-FETs that each connect with an electronic circuit. The same body fluid sample is tested, and the signal change from each g-FET indicates if the test subject potentially has a disease that is indicated by the presence of the multiple biomarkers in the body fluid sample. With multiple biomarkers separated tested for on individual g-FETs from the same bio-fluid sample, the likelihood the test subject has the target disease (pancreatic cancer) can be determined through a single and simple diagnostic test.

To selectively immobilize the multiple different capture molecules onto the different charge transfer layers, a capture molecule carrier fluid is disposed over the surface of the semiconductor substrate wafer covering the plurality of device regions (step two). The capture molecule carrier fluid contains N sets of activatable capture molecules provided as free-floating activatable capture molecules. Each respective set of activatable capture molecules is activated for binding by a different corresponding wavelength of capture molecule-activating radiation.

The surface of the semiconductor wafer is selectively radiated with a pattern of radiation that includes a first wavelength of linker-activating radiation 5302 and a first wavelength of capture molecule-activating radiation 5304 to activate and bind a first set of activated capture molecules 5214 to a first set of activated linker molecules 5220. Alternatively, the chemistries of the constitute molecules can be such that selective irradiation of only the linker or capture molecule is needed for the selective binding to occur.

In this embodiment, a shadow mask 4902 is used to selectively pattern wavelengths of linker-activating and capture molecule-activating radiation (step three). The charge transfer layers of two or more adjacent device regions has two or more immobilized linker molecules that are each activated by a different wavelength of radiation. The capture molecule carrier fluid has two or more corresponding capture molecules that become activated by a different wavelength of radiation. As shown, the first wavelength of linker-activating radiation 5302, and the first wavelength of capture molecule-activating radiation 5304 make a matched pair of both a first set of activated capture molecules 5214 and corresponding first set of activated linker molecules 5220 immobilized on the charge transfer layer that is being irradiated. An adjacent device region also has the two or more different linker molecules randomly immobilized on that charge transfer layer, and above the immobilized linker molecules the capture molecule carrier fluid has the same randomly distributed different types of capture molecules. But the shadow mask blocks radiation from reaching these linker and capture molecules so no binding occurs. Each of the activated linker molecules 5220 and activated capture molecules 5214 become receptive for binding only if irradiated with their specific wavelength of radiation. Once irradiated as shown in step three, the activated linker and capture molecules that are made receptive to binding will only bind with each other. The other linker molecules immobilized on the charge transfer layer do not bind with the other free-floating capture molecules. By this process, each device region can be functionalized with a selected capture molecule or more than one selected different type of capture molecules. The set of corresponding binding wavelengths can be applied in sequence, or simultaneously, and applied as a pulsed beam or flooded onto a mask to form a pattern of different capture molecules immobilized on different g-FETs.

FIG. 54 illustrates the steps for selectively binding a second set of activated capture molecules to a second set of activated linker molecules, to form a bare die semiconductor device with two or more separately functionalized g-FET biosensors. The resulting bare die g-FETs can be packaged for printed circuit board use, or as a component part of a larger integrated circuit or system-on-a-chip.

After the selective functionalization of the first set of capture molecules, the wafer has some of the g-FET devices that are now functionalized and other g-FET devices that only have the linker molecules immobilized on the charge transfer layer (step four). The shadow mask is disposed so that the functionalized g-FETs are protected from further functionalization by blocking the next applied wavelengths of radiation. Subsequently, the surface of the semiconductor wafer can be irradiated with a second pattern of radiation that includes the second wavelength of linker-activating radiation and the second wavelength of capture molecule-activating radiation to bind a second set of activated capture molecules 5216 to a second set of activated linker molecules 5222. To cause the selective binding, a second wavelength of capture molecule-activating radiation 5404 and a second wavelength of linker-activating radiation 5406 are irradiated through the shadow mask to that only the desired g-FET devices are functionalized (step five).

By this process of selective patterning of predetermined wavelengths of radiation, the semiconductor wafer can be functionalized so that it has adjacent g-FET devices that are each functionalized to detect a different biomarker. By combining the adjacent g-FET devices into a signal biosensor, multiple biomarker sensor bare dies 5402 are formed with the multiple functionalization steps all performed at the wafer-level (step six).

The source, drain, gate, electrodes, etc., features formed on the semiconductor wafer, and the resulting multiple biomarker sensor bare dies 5402 diced from the wafer can then be ready to be wire bonded and packaged (step seven). For example, multiple g-FETs can share a common drain 3204, and/or the g-FETs can be formed in serial and/or parallel connection sharing common source, drain and/or gate electrodes.

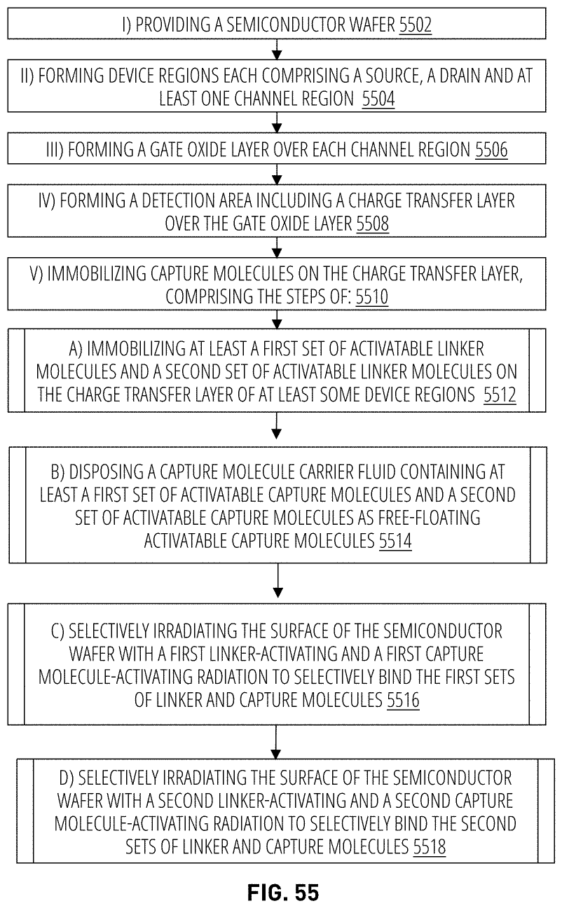

FIG. 55 is a flow chart showing the steps for selectively immobilizing different respective capture molecules on different corresponding g-FETs. In block 5502, a semiconductor wafer is provided. Device regions are formed, each comprising a source, a drain and at least one channel region (block 5504). A gate oxide layer over each channel region (block 5506).

The gate oxide layer can be a gate oxide stack comprising at least one oxide that has a lower dielectric constant, K, with relatively less ability to store electrical energy and is more of an insulator layer and at least one oxide that has a higher K and is more of a dielectric layer with relatively greater ability to store electric energy in an electrical field. The gate oxide stack can be tuned to a particular set of parameters for the intended use-cases for the biosensor. The tuning takes into consideration device construction and use-case parameters including at least one of: sample medium, type of capture molecules, Debeye screening length, voltage and current during use, etc.

In block 5508, a detection area is formed including a charge transfer layer formed over the gate oxide layer. In block 5510 capture molecules are immobilized on the charge transfer layer. The immobilization of the capture molecules comprises the steps of: a) immobilizing at least a first set of activatable linker molecules and a second set of activatable linker molecules on the charge transfer layer of device region, each respective first set and second set of activatable linker molecules being activated for binding by a different corresponding first wavelength of linker-activating radiation and second wavelength of linker-activating radiation (block 5512). In block 5514, a capture molecule carrier fluid is disposed over the surface of the semiconductor substrate wafer covering the plurality of device regions. The capture molecule carrier fluid contains different sets of activatable capture molecules as free-floating activatable capture molecules. Each respective set of activatable capture molecules is activated for binding by a different corresponding wavelength of capture molecule-activating radiation. In block 5516, the surface of the semiconductor wafer is irradiated with a first pattern of radiation comprising the first wavelength of linker-activating radiation and the first wavelength of capture molecule-activating radiation to bind a first set of activated capture molecules to a first set of activated linker molecules on the first set of g-FETs. In block 5518, the surface of the semiconductor wafer is selectively irradiated with a second pattern of radiation comprising the second wavelength of linker-activating radiation and the second wavelength of capture molecule-activating radiation to bind a second set of activated capture molecules to a second set of activated linker molecules on the second set of g-FETs.

This process of selective functionalization of different g-FETs with different capture molecules can be done using conventional semiconductor fabrication techniques such as spin and dip coating, patterned radiation through masks, etc. Also, pulsed and focused wavelengths of radiation (e.g., laser beam) can be scanned over the surface of the semiconductor wafer in addition to or in replacement for flooding radiation through a shadow masks. The g-FET semiconductor devices formed on the semiconductor wafer can be functionalized so that adjacent g-FET devices that are each functionalized to detect a different biomarker, and adjacent g-FET devices functionalized for detecting different biomarkers from the same biosample can be combined into a single packaged biosensor, with the fabrication and functionalization steps performed in the near pristine environment of semiconductor fabrication at the wafer-level.

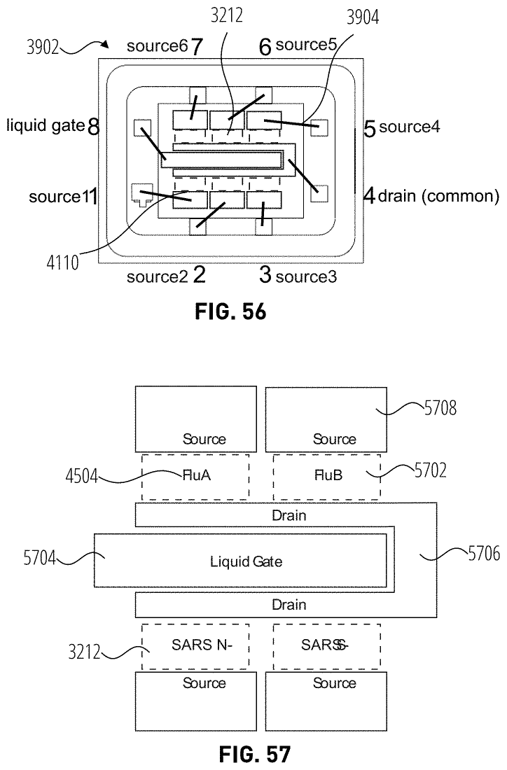

FIG. 56 illustrates a single bare die wire bonded for semiconductor packaging and having multiple and differently functionalized g-FET biosensors. The charge transfer layer 3212 of each g-FET 4110 on the bare die semiconductor device is functionalized with a different set of capture molecules. A DIP package 3902 is constructed using conventional semiconductor electronic circuit device materials and processes including wire bonds 3904 connecting the electrodes of the g-FET biosensor features (sources, drains, liquid gate) to respective pins of the DIP package. An encapsulation process encases the bare die and leaves an opening so that the detection areas of the g-FETs are accessible by the sample being tested.

FIG. 57 illustrates a bare die biosensor having g-FET devices functionalized for FluA/FluB/SARS virus testing. In this case, each g-FET on the bare die semiconductor device is functionalized for detecting if the test sample contains a biomarker of FluA, FluB or SARS. In this example, the SARS biomarkers include both the SARS N-protein and S-protein. Each charge transfer layer 3212 of the different g-FETs has a different type of capture molecule (e.g., capture molecule1 4504 for detecting FluA biomarker). The capture molecules are immobilized at the detection area 5702 of each corresponding g-FET. A liquid gate electrode 5704, a drain electrode 5706, and a source electrode 5708 provide electrical conduction to the semiconductor features that form the different biosensors ganged on on semiconductor bare die, where one or more of these biosensors can be functionalized at the wafer level.

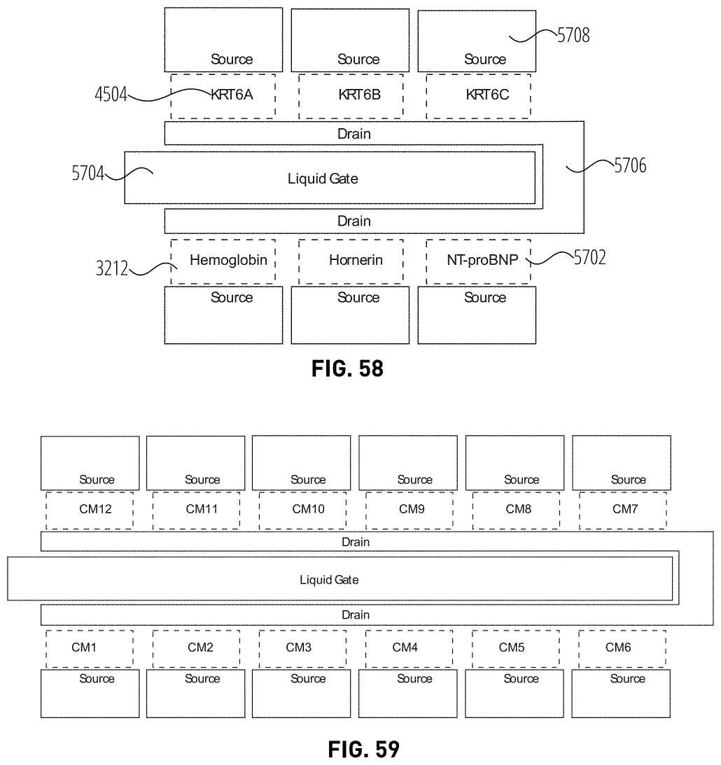

FIG. 58 illustrates a bare die biosensor having multiple and differently functionalized g-FET biosensors for detecting lung cancer. There is a tremendous need for a simple, non-invasive, and low cost screening test for lung cancer. In the US, Lung cancer is the largest cause of cancer deaths, each year 200,000 are diagnosed and 150,000 die from lung cancer. Additionally, smokers die from other smoking-related diseases, including heart disease, emphysema and stroke. For lung cancer, the diagnosis is typically too late for long-term survival prognosis, in large part due to the cost and difficulty of the diagnosis. The CDC says the only recommended screening test for lung cancer is computer tomography, an expensive test requiring specialized and costly equipment and a high degree of technical expertise from skilled technicians and medical doctors. The result is that lung cancers that could be treated successfully are often overlooked and left untreated.

In accordance with this non-limiting exemplary embodiment, a bare die semiconductor device is fabricated with wafer level functionalization for detecting six different biomarkers. Five of the biomarkers are specific to lung cancer and have been identified as being present in EBC. Functionalization for a sixth biomarker is provided for as well, where a biomarker known to be present in EBC and indicative of heart disease is tested for along with the lung cancer biomarkers. Since smokers often die from smoking related diseases including heart disease, emphysema and stroke, testing the EBC of a smoker through a simple-to-use and low cost mask-based diagnostic device can provide an early detection of lung cancer and/or other smoking-related health concerns. The biomarkers and number of different g-FETs is by way of example, there could be more or less different biomarkers tested, and this example configuration of a multi-biomarker biosensor can be applied to test for other diseases using other bio-samples, such as pancreatic cancer tested for using GI lavage as the bio-sample.

On the charge transfer layer 3212 of each g-FET ganged on the bare die, a different capture molecule (e.g., capture molecule1 4504) is immobilized at the detection area 5702. The liquid gate electrode 5704 and the drain electrode 5706 can be common for all, or one or more, of the g-FETs, and a separately connected source electrode 5708 for each g-FET enables the same electronics to scan each biosensor in a sequence of signal sampling. The combination of test result signals from different biomarkers related to the same disease provides a greater statistical accuracy for the diagnosis, and/or can be used to rule out a suspected disease.

EBC has been demonstrated to contain biomarkers that can be used to detect lung cancer early. In accordance with a non-limiting, exemplary embodiment, a mask-based diagnostic device includes a biosensor with multiple and differently functionalized g-FET biosensors. The biosensors are functionalized to detect lung cancer biomarkers, such as cytokeratins, hemoglobin and hornerin. Researchers have found cytokeratins (KRTs) including KRT6A, KRT6B, and KRT6C isoforms, were significantly elevated in EBC taken from lung cancer patients. The amount of KRTs in EBC samples from lung cancer patients also showed significant positive correlation with tumor size. Hemoglobin and Hornerin were also frequently elevated in EBC of patients with lung cancer. Researchers have also verified that NT-proBNP found in EBC is an indicator of heart disease.

In accordance with this exemplary embodiment, g-FETs are differently functionalized on the same bare die. In the example of a lung cancer screening test, the g-FETs can be functionalized with KRT6A, KRT6B, KRT6C, Hemoglobin and Hornerin capture molecules. In the case of the biosensor being part of the mask-based diagnostic device, the same EBC biosample collected by the EBC collector inside the face mask flows over the detection area of the biosensor on the EBC testing unit. The same electronics of the printed circuit board of the EBC testing unit can detect, analyze and wirelessly transmit the test results depending on whether the target molecules (KRT6A, KRT6B, KRT6C, Hemoglobin and Hornerin) are capture by the respective capture molecules immobilized on the charge transfer layer of the corresponding five g-FETs. Also, the same EBC sample can be tested for biomarkers of another disease or health condition, such as heart disease, by NT-proBNP capture molecules immobilized on a sixth g-FET formed on the bare die.

FIG. 59 illustrates a bare die biosensor having twelve functionalized g-FET biosensors for detecting one or more diseases, health conditions and/or environmental conditions. This exemplary embodiment shows that many differently functionalized g-FET biosensor semiconductor devices can be formed and functionalized on the same wafer. As an example, a panel test that can detect a number of different biomarkers can be configured for use in a mask-based diagnostic device, or a table top testing system, and be used to screen for multiple different possible diseases and health conditions. For example, the patient demographics, medical records, family history, lifestyle, etc., can be used to select a panel of biomarkers for use at a yearly doctor examination, providing a proactive testing regimen for the patient. A panel of biomarkers in a mask-based diagnostic can used as to screen apparently healthy healthcare workers at a hospital for non-symptomatic viruses that could infect less healthy patients. As countries learn how to live with the SARS-CoV-2 virus, a panel test for Flu1/FluB/SARS can be used to quickly determine the state of infectivity and type of virus a person has before entering a stadium, school, or boarding an airplane.

Acute kidney injury (AKI) is one of several possible health conditions for an elderly patient complaining of chest pain, fatigue, shortness of breath and confusion. The outcomes of acute kidney injury (AKI) could be severe and even lethal, if not diagnosed in its early stages and treated appropriately. Blood and urine biomarkers, currently in use as indicators for kidney function, are either inaccurate or not timely. However, there is a dramatic change in exhaled breath composition associated with kidney dysfunction after ischemic insult. For example, elevated levels of neutrophil gelatinase-associated lipocalin (NGAL) is a biomarker for early diagnosis and prediction for acute kidney injury (AKI). Capture molecules for NGAL and/or other AKI specific biomarkers can be functionalized on the biosensor to indicate the potential of AKI for a patient complaining of chest pain, fatigue, shortness of breath and confusion. However, thrombosis can also cause these same symptoms in an elderly patient. Elevated levels of D-dimer present in EBC is a biomarker for diagnosis of thrombosis. On the other hand, stroke and heart disease can also cause these symptoms, and erythropoietin present in EBC is a biomarker for stroke, and NT-proBNP is a biomarker present in EBC for diagnosing a heart attack.

The mask-based diagnostic with a multi-biomarker panel test biosensor system can be a great advantage for diagnosis where time is of the essence. The biomarkers that are tested for can be selected depending on the demographics and typical presenting symptoms of the test subject. For example, consider the situation where an elderly resident at a nursing home complains of chest pain, fatigue, shortness of breath and confusion. An ambulance is called to rush the patient to the hospital. The attending paramedics arrive and immediately begin checking the patient vital signs and symptoms, put the patient on a gurney, etc. After stabilizing the patient, a mask-based diagnostic device is put on the patient with the multi-biomarker panel test indicated for the patient demographics and major symptoms. The patient only has to breathe for the mask-based diagnostic system to quickly test for a range of biomarkers for diseases and health conditions that are consistent with the same symptoms.

On the way to the hospital, after about 10 minutes of wearing the mask-based diagnostic, the paramedic attending to the patient in the back of the ambulance is alerted that the test results on the levels of the biomarkers tested for in the EBC of the patient indicate that most likely the patient is experiencing AKI. A smartphone APP communicates to the paramedic that prompt reversal of fluid deficits is critical in preventing further exacerbation of AKI, and that the patient should be given intravenous isotonic solutions (e.g., normal saline) instead of hyperoncotic solutions (e.g., dextrans, hydroxyethyl starch, albumin). The APP also alerts the hospital that the patient will be coming to the emergency room with a possible AKI diagnosis and appropriate tests should be readied to immediately confirm or correct the diagnosis, including renal ultrasonography to rule out obstruction. The patient arrives at the hospital, the appropriate tests are immediately performed confirming the diagnosis of AKI, making possible the rapid interventions that save the patient's life.

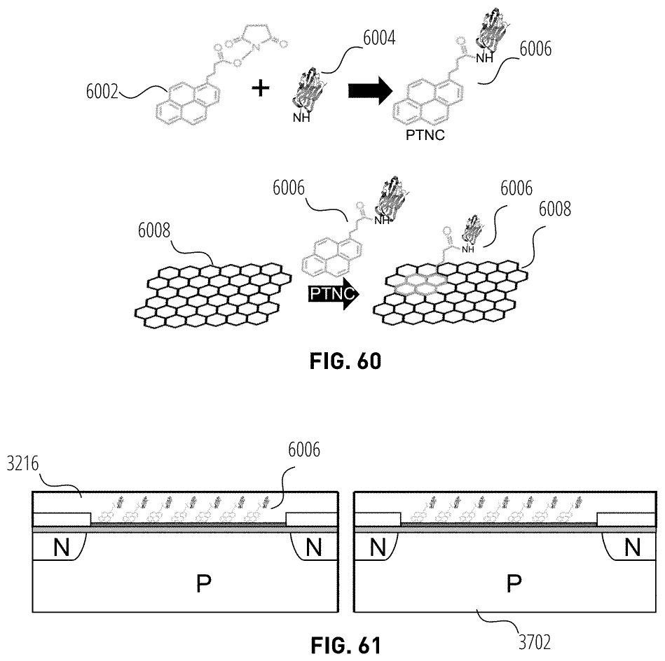

FIG. 60 illustrates the cross-linking of pyrenebutyric acid N-hydroxysuccinimide ester (PBASE 6002) with an aminated nanoCLAMP 6004 capture molecule to form a pyrene-tagged nanoCLAMP (PTNC 6006) capture molecule conjugate, and then a one-step process for immobilizing the PTNC capture molecule conjugate on a graphene 6008 charge transfer layer.

An aqueous functionalization process for immobilizing linker/capture molecule conjugates at the wafer level before separating the bare die from the wafer has the advantage of semiconductor fabrication described herein, with added advantages of fewer wafer level processing steps and avoiding the typically used strong solvents dimethyl formamide (DMF) or dimethyl sulfoxide (DMSO) coming in contact with any of the semiconductor materials. If functionalization is performed at the device level, aqueous functionalization may allow for the use of conventional encapsulation materials, which might otherwise dissolve in a strong organic solvent, such as dimethylformamide (DMF) or dimethyl sulfoxide (DMSO).

Researchers have recently developed a one-step aqueous functionalization process for immobilizing aptamer capture molecules onto a graphene charge transfer layer of a g-FET sensor (see, Khan, N. I.; Song, E., Detection of an IL-6 Biomarker Using a GFET Platform Developed with a Facile Organic Solvent-Free Aptamer Immobilization Approach. Sensors, 2021, 21, 1335). In accordance with a non-limiting, exemplary embodiment bare die g-FET sensors are functionalized at the wafer level using a one-step aqueous functionalization process.

To form pyrene-tagged nanoCLAMPs (PTNCs), pyrene groups (PBASE 6002) are first cross-linked to amine-terminated nanoCLAMPs 6004. The cross linking can be accomplished by incubating the aminated nanoCLAMPs with PBASE dissolved in DMF. DMF is a strong solvent that could degrade semiconductor features, such as passivation layers, if used in a wafer-level process. In accordance with this embodiment, the linker/capture molecule conjugates (e.g., PTNCs) can be formed by solution processing in a processing reactor outside of the processing of the semiconductor wafer, to prevent any contact of the solvent, like DMF or DMSO, on the semiconductor wafer. Once the linker/capture molecule conjugates are formed, the PTNCs can be purified by precipitation with the help of a centrifuge. As described in more detail below, the purified PTCNs can then be put into a water-based carrier fluid and disposed into the detection well (shown in FIG. 25) for an incubation process to immobilize the capture molecules on the g-FET charge transfer layer(s) of semiconductor g-FET(s) in a packaged semiconductor device.

FIG. 61 illustrates bare die g-FET biosensors diced from the wafer after the one-step process for immobilizing the PTNC capture molecule conjugate and the formation of a protection layer at the wafer level. At the wafer level, linker/capture molecule conjugates (e.g., pyrene-tagged nanoCLAMPS PTCNs 6006) are immobilized onto the graphene charge transfer layers of all the g-FETs formed on the wafer, then a protection layer 3216 is formed over the wafer. The protection layer can comprise a relatively thin, crystalline and water-soluble material, such as sucrose, that is formed from an aqueous solution, spin coated onto the wafer, and then the water evaporated. The hardened, crystalline sucrose layer surrounds and locks in place the location and orientation of the immobilized linker/capture molecule conjugates. The protection layer can be patterned at the wafer level, for example, using patterned x-y axis scanned laser ablation, where the crystalline sucrose is selectively removed from the bonding pads (electrodes) and other areas of the wafer, while leaving the protection layer in place protecting the immobilized capture molecules. Alternatively, a mask can be formed to cover portions to be left in place so that the protection layer can be selectively patterned by a water rinse, or plasma etched. After forming and processing the various semiconductor features, functionalized charge transfer layers, and protection layer at the wafer level, the wafer is diced into bare die 3702. If the protection layer is not patterned and left in place over the bonding pads, a subsequent selective removal process may be performed to expose the bonding pads prior to wire bonding, or during the wire bonding process, the thin, crystalline protection layer may be scrubbed away by the action of wire bonding, as the wire and bonding pad surfaces are brought into intimate contact in a solid phase welding process, such as ultrasonic welding.

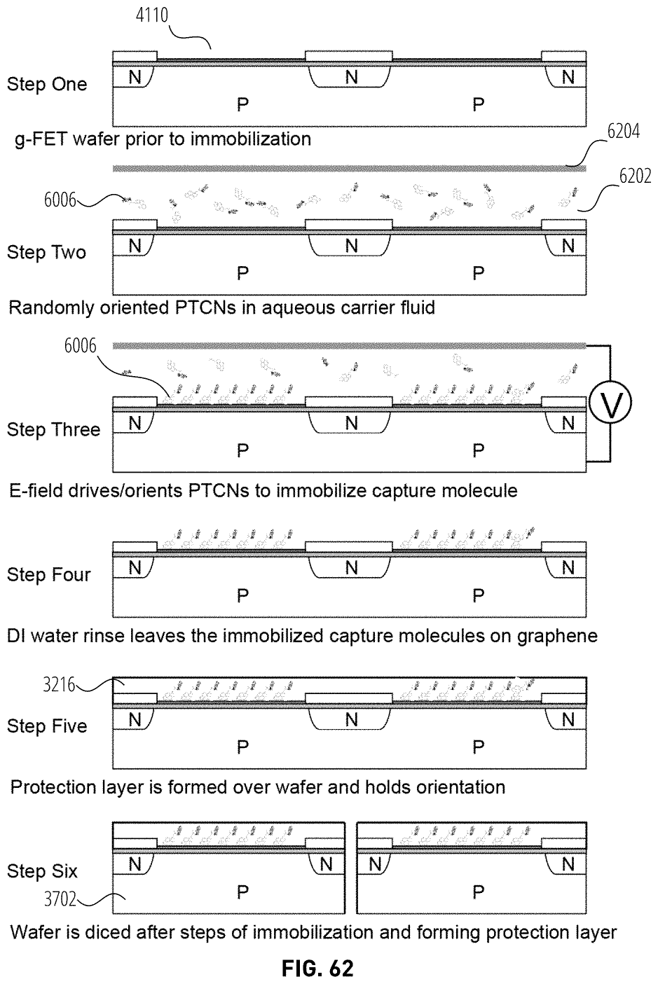

FIG. 62 illustrates the steps of making a semiconductor wafer of graphene field effect transistor biosensors with a one-step process for immobilizing PTNC capture molecule conjugates and the formation of a protection layer at the wafer level.

In step one, a semiconductor wafer is provided having g-FETs 4110 formed where each device includes a source, a drain and at least one channel region, and a gate oxide layer formed over each channel region. A detection area includes a charge transfer layer formed over the gate oxide layer. Capture molecules are immobilized on the charge transfer layer by first disposing randomly dispersed linker/capture molecule conjugates (PTNCs 6006) in an aqueous carrier fluid 6202 on the entire top surface of the semiconductor wafer or covering at least a portion of the device regions. A driving electrode 6204 is brought into electrical contact with the carrier fluid (step two). Preferably, the driving electrodes is a planar electrode that has a surface area matching the surface of the wafer containing the device regions that are to be functionalized.

The linker/capture molecule conjugate is a dipole conjugate molecule each having a linker end of one polarity and a capture molecule end of another polarity. A voltage is applied to the driving electrode in electrical contact with the aqueous carrier fluid and to the semiconductor wafer to apply an electrical aligning field in the aqueous carrier fluid (step three). The electrical aligning field orients and drives the linker/capture molecule conjugates toward the charge transfer layer to bind a linker end to the charge transfer layer and immobilize the capture molecule end on the graphene charge transfer layer (step four). A protection layer 3216 may be formed at least over the detection area to protect and maintain the orientation and location of the immobilized capture molecules (step five). After immobilizing the capture molecules, individual semiconductor devices are separated from the semiconductor wafer (step six).

As alternatives, the linker and capture molecules can be separately immobilized sequentially and in different carrier fluids, and the driving electrode and applied voltage used to orient and drive either or both in the immobilization sequence.

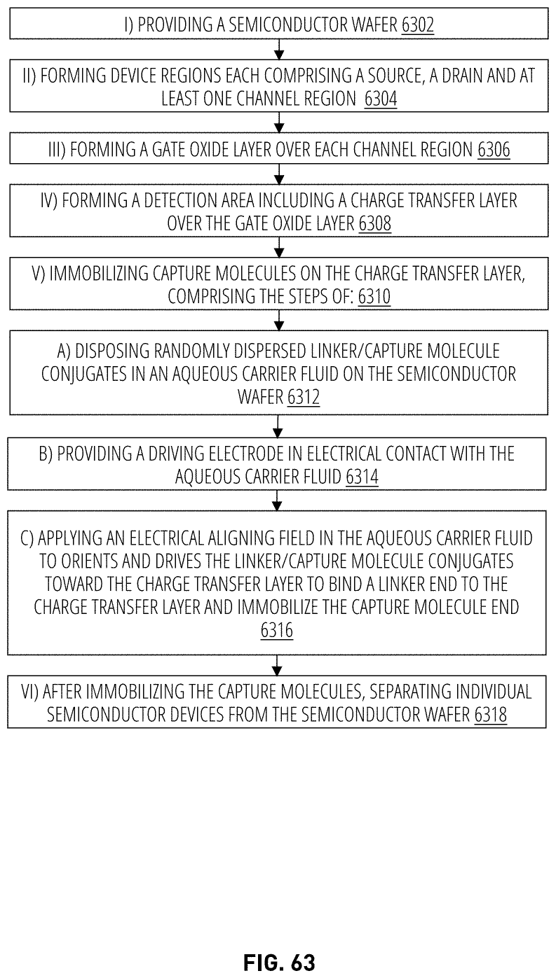

FIG. 63 is a flowchart of the steps for making a semiconductor wafer of g-FET biosensors where capture molecule conjugates are immobilized on graphene charge transfer layers at the wafer level.

In block 6302, a semiconductor wafer. In block 6304, g-FET device regions are formed each comprising a source, a drain and at least one channel region. In block 6306, a gate oxide layer is formed over each channel region. In block 6308, a detection area is formed including a charge transfer layer over the gate oxide layer. In block 6310, capture molecules are immobilized on the charge transfer layer, where in block 6312, a) randomly dispersed linker/capture molecule conjugates are disposed in an aqueous carrier fluid on the top surface of the semiconductor wafer and covering at least a portion of the device regions. The linker/capture molecule conjugates each have a linker end of one polarity and a capture molecule end of another polarity. In block 6314, b) a driving electrode is provided in electrical contact with the aqueous carrier fluid. In block 6316, c) a voltage is applied to the driving electrode and the semiconductor wafer to apply an electrical aligning field in the aqueous carrier fluid. The electrical aligning field orients and drives the linker/capture molecule conjugates toward the charge transfer layer to bind a linker end to the charge transfer layer and immobilize the capture molecule end. In block 6318, vi) after immobilizing the capture molecules, individual semiconductor devices are separated from the semiconductor wafer.

Various modifications and adaptations to the foregoing exemplary embodiments of this invention may become apparent to those skilled in the relevant arts in view of the foregoing description, when read in conjunction with the accompanying drawings. However, any and all modifications will still fall within the scope of the non-limiting and exemplary embodiments of this invention.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

1-16. (canceled)

17. A method for making a semiconductor biosensor, comprising the steps of:
i) providing a semiconductor substrate wafer of one conductivity type;
ii) forming a plurality of semiconductor device regions in the semiconductor substrate wafer, each semiconductor device region comprising at least a source region and a drain region defining there between a channel region of the one conductivity type with the source region and the drain region of an opposite conductivity type;
iii) forming a detection area over the channel region, the detection area including a charge transfer layer;
iv) immobilizing capture molecules on the charge transfer layer of at least a portion of the plurality of semiconductor device regions; and
v) separating individual semiconductor devices from the semiconductor substrate wafer, each comprising at least one said semiconductor device region of the plurality of semiconductor device regions, wherein the step of separating is performed after the step immobilizing.

18. The method of claim 17, further comprising a step of forming an insulator layer over the channel region prior to iii) forming the detection area.

19. The method of claim 17, further comprising a step of forming a dielectric layer over the channel region prior to iii) forming the detection area.

20. The method of claim 17, wherein the charge transfer layer comprises a graphene layer.

21. The method of claim 17, further comprising a step of forming a protection layer over the charge transfer layer after the step of iv) immobilizing the capture molecules and before the step of separating the individual semiconductor devices.

22. The method of claim 17, further comprising a step of forming a protection layer over the charge transfer layer to protect the immobilized capture molecules, wherein the protection layer is removable by a solvent after the step of separating without removing the immobilized capture molecules from the charge transfer layer.

23. The method of claim 17, wherein the step of iv) immobilizing the capture molecules comprises immobilizing a first type of capture molecule on the charge transfer layer of each of a first sub-set of the plurality of semiconductor device regions and immobilizing a second type of capture molecule on the charge transfer layer of each of a second sub-set of the plurality of semiconductor device regions.

24. The method of claim 23, further comprising a step of forming a protection layer pattern over the charge transfer layer of said each of the first sub-set prior to immobilizing the capture molecules.

25. The method of claim 24, wherein the protection layer is sucrose.

26. The method of claim 17, wherein the step of iv) immobilizing capture molecules on the charge transfer layer, comprises the steps of:
a) immobilizing activatable linker molecules on the charge transfer layers;
b) disposing a capture molecule carrier fluid containing the capture molecules as free-floating capture molecules over a top surface of the semiconductor substrate wafer covering the plurality of device regions, where prior to activation the activatable linker molecules are relatively less receptive to binding to the free-floating capture molecules;
c) selectively activating the activatable linker molecules to form activated linker molecules immobilized at some of the charge transfer layers, the activated linker molecules binding with the free-floating capture molecules; and
d) binding the capture molecules to the activated linker molecules.

27. A method, comprising the steps of:
i) providing a semiconductor wafer;
ii) forming device regions comprising a source, drain and a channel region;
iii) forming at least one of an insulator layer and a dielectric layer over at least the channel region;
iv) forming a detection area including a charge transfer layer over said at least one of an insulator layer and a dielectric layer;
v) immobilizing capture molecules on the charge transfer layer; and
vi) after immobilizing the capture molecules separating individual semiconductor devices from the semiconductor wafer.

28. The method of claim 27, wherein the step of v) immobilizing comprises providing linker molecules in a linker carrier fluid and immobilizing the linker molecules on the charge transfer layers in a first incubation step; and then providing the capture molecules in a capture molecule carrier fluid and binding the capture molecules to the linker molecules in a second incubation step.

29. The method of claim 28, wherein the charge transfer layer comprises at least one of monolayer of graphene, hexagonal boron nitride (h-BN), silicene, germanium, black phosphorus (BP) and transition metal sulfides.

30. The method of claim 27, wherein the step of v) immobilizing comprises providing the capture molecules as polarized capture molecule conjugates including a linker molecule end and capture molecule end, and applying an electrostatic field to orient and drive the linker molecule end to facilitate binding the linker molecule end with the charge transfer layer.

31. The method of claim 27, further comprising forming a protection layer over at least the charge transfer layers after the stop of (v) immobilizing.

32. The method of claim 31, where the protection layer comprises sucrose.

33. A method, comprising the steps of:
i) providing a semiconductor wafer;
ii) forming device regions each comprising a source, a drain and at least one channel region;
iii) forming at least one of an insulator layer and a dielectric layer over each channel region;
iv) forming a detection area including a charge transfer layer over said at least one of an insulator layer and a dielectric layer;
v) immobilizing capture molecules on the charge transfer layers, comprising the steps of
a) immobilizing activatable linker molecules on the charge transfer layers; and
b) disposing a capture molecule carrier fluid containing the capture molecules as free-floating capture molecules over a top surface of the semiconductor substrate wafer covering the plurality of device regions, where prior to activation the activatable linker molecules are relatively less receptive to binding to the free-floating capture molecules;
c) selectively activating the activatable linker molecules to form activated linker molecules immobilized at some of the charge transfer layers, the activated linker molecules binding with the free-floating capture molecules; and
d) binding the capture molecules to the activated linker molecules.

34. The method of claim 33, further comprising the step of vi) After immobilizing the capture molecules separating individual semiconductor devices from the semiconductor wafer.

35. The method of claim 33, further comprising the step of forming a protection layer over at least the charge transfer layers after the step of immobilizing the capture molecules on the charge transfer layers

36. A method, comprising the steps of:
i) providing a semiconductor wafer;
ii) forming device regions each comprising a source, a drain and at least one channel region;
iii) forming a gate oxide layer over each channel region;
iv) forming a detection area including a charge transfer layer over the gate oxide layer;
v) immobilizing capture molecules on the charge transfer layers, comprising the steps of
a) immobilizing at least a first set of activatable linker molecules and a second set of activatable linker molecules on the charge transfer layer of device region, each respective first set and second set of activatable linker molecules being activated for binding by a different corresponding first wavelength of linker-activating radiation and second wavelength of linker-activating radiation; and
b) disposing over a surface of the semiconductor substrate wafer covering the plurality of device regions a capture molecule carrier fluid containing at least a first set of activatable capture molecules and a second set of activatable capture molecules as free-floating activatable capture molecules, each respective first set and second set of activatable capture molecules being activated for binding by a different corresponding first wavelength of capture molecule-activating radiation and second wavelength of capture molecule-activating radiation;
c) selectively irradiating the surface of the semiconductor wafer with a first pattern of radiation comprising the first wavelength of linker-activating radiation and the first wavelength of capture molecule-activating radiation to bind a first set of activated capture molecules to a first set of activated linker molecules;
d) selectively irradiating the surface of the semiconductor wafer with a second pattern of radiation comprising the second wavelength of linker-activating radiation and the second wavelength of capture molecule-activating radiation to bind a second set of activated capture molecules to a second set of activated linker molecules.

37. The method of claim 36, wherein the first wavelength of linker-activating radiation and the first wavelength of capture molecule-activating radiation are the same wavelength.

38. The method of claim 31, further comprising a step of forming a protection layer pattern over at least the charge transfer layers after the step of immobilizing capture molecules on the charge transfer layers.

39. The further of claim 38, wherein the protection layer comprises sucrose.

40. A method, comprising the steps of:
i) providing a semiconductor wafer;
ii) forming device regions each comprising a source, a drain and at least one channel region;
iii) forming a gate oxide layer over each channel region;
iv) forming a detection area including a charge transfer layer over the gate oxide layer;
v) immobilizing capture molecules on the charge transfer layer, comprising the steps of
a) disposing randomly dispersed linker/capture molecule conjugates in an aqueous carrier fluid or a top surface of the semiconductor wafer and covering at least a portion of the device regions, the linker/capture molecule conjugates each having a linker end of one polarity and a capture molecule end of another polarity;
b) providing a driving electrode in electrical contact with the aqueous carrier fluid;
c) applying a voltage to the driving electrode and the semiconductor wafer to apply an electrical aligning field in the aqueous carrier fluid, wherein the electrical aligning field orients and drives the linker/capture molecule conjugates toward the charge transfer layer to bind a linker end to the charge transfer layer and immobilize the capture molecule end; and
vi) after immobilizing the capture molecules, separating individual semiconductor devices from the semiconductor wafer.
